(19) United States
(12) Patent Application Publication
(10) Pub. No.: US 2006/0160721 A1
(43) Pub. Date: Jul. 20, 2006

(54) Method of plasma lipidation to prevent, inhibit and/or reverse atherosclerosis

(75) Inventor: Henry J. Pownall, Houston, TX (US)

Correspondence Address: FULBRIGHT & JAWORSKI, LLP, 1301 MCKINNEY, SUITE 5100, HOUSTON, TX, 77010-3095, US

(73) Assignee: BAYLOR COLLEGE OF MEDICINE, Houston, TX (US)

(21) Appl. No.: 11/316,062

(22) Filed: Dec. 22, 2005

Related U.S. Application Data
(60) Provisional application No. 60/638,906, filed on Dec. 22, 2004.
(60) Provisional application No. 60/675,825, filed on Apr. 28, 2005.

(51) Int. Cl. A61K 38/17; C12Q 1/60; A61K 31/685

(52) U.S. Cl. 514/2; 514/78; 435/11

(57) ABSTRACT

The present invention relates to a composition comprising a detergent that can alter the activity of a lipoprotein. Additional aspects include the composition comprising a phospholipid, and the use of the compositions to treat and/or prevent cardiovascular diseases.

### CROSS-REFERENCE TO RELATED APPLICATIONS

This application claims priority to U.S. Provisional Application No. 60/638,906 filed Dec. 22, 2004 and U.S. Provisional Application No. 60/675,825 filed Apr. 28, 2005, each of which is incorporated herein by reference in its entirety.

### STATEMENT REGARDING FEDERALLY SPONSORED RESEARCH OR DEVELOPMENT

This invention was made in part with government support under Grant No. R01 HL-56865 and R01 HL-30914-12 awarded by the National Institutes of Health. The United States Government may have certain rights in the invention.

### TECHNICAL FIELD

The present invention relates to a method of altering the structure of a lipoprotein thereby altering or enhancing the activity associated with a lipoprotein, for example cholesterol binding affinity or cholesterophilicity. The methods comprise administering to a blood sample a composition comprising detergent and/or phospholipid.

### BACKGROUND OF THE INVENTION

Coronary heart disease (CHD) remains the leading cause of death in the industrialized countries. The primary cause of CHD is atherosclerosis, a disease characterized by the deposition of lipids, including cholesterol, in the arterial vessel wall, resulting in a narrowing of the vessel passages and ultimately hardening of the vascular system and a life-threatening restriction of blood flow.

Atherosclerosis generally begins with local injury to the arterial endothelium followed by proliferation of arterial smooth muscle cells from the medial layer to the intimal layer along with the deposition of lipid and accumulation of foam cells in the lesion. As the atherosclerotic plaque develops it progressively occludes more and more of the affected blood vessel lumen and can eventually lead to ischemia or infarction. Because deposition of circulating lipids such as cholesterol plays a major role in the initiation and progression of atherosclerosis, it is important to identify compounds, methods and compositions to help remove cholesterol from the developing peripheral tissues, including atherosclerotic plaque.

Unlike the liver, extrahepatic tissue can synthesize cholesterol, but cannot degrade it. Thus, cholesterol accumulation in macrophages, a key cell type in atherogenesis, produces a pathological state, unless there is an effective mechanism for cholesterol disposal. Such a mechanism has been described as reverse cholesterol transport (RCT). RCT comprises three steps: cellular cholesterol efflux from peripheral tissues to various early forms of HDL; remodeling of early forms of HDL in the plasma compartment; and uptake of lipid in mature forms of HDL and low density lipoproteins (LDL) by hepatic receptors.

In human plasma, early forms of lipoproteins are remodeled by multiple enzymes and transfer proteins that convert them to mature particles that are recognized by cell surface receptors that mediate their uptake and catabolism (Rye et al, 2002, 2004; Nakamura et al, 2004). Lipoprotein lipase converts very low density lipoproteins (VLDL) to intermediate density lipoproteins, which are further lipolyzed by hepatic lipase giving LDL, which are removed by hepatic LDL-receptors. Early forms of high density lipoproteins (HDL), which receive cholesterol via interactions with ABC transporters in peripheral tissue (Oram, 2002; Wang et al, 2004; Nakamura et al, 2004), are substrates for lecithin:cholesterol acyltransferase (LCAT), which esterifies HDL-cholesterol while converting HDL to a mature form that is recognized by hepatic HDL receptors (Webb et al, 2004). This process, reverse cholesterol transport is an important component of normal lipid metabolism and a potential therapeutic target. Thus, characterization of the structure, dynamics, and stability of plasma lipoproteins is an important key to understanding how interacting biomolecules determine function.

Although physical techniques that include calorimetry, mass spectrometry, nuclear magnetic resonance, X-ray crystallography, and fluorescence spectroscopy have been used to study biomolecular structures, these methods are sometimes inadequate, and less direct methods of chemical perturbation such as protein denaturation or proteolytic and lipolytic probes of structure and stability must be used; this is particularly true for complex assemblies of lipids and proteins that are found in membranes and plasma lipoproteins. Some of these methods are still overly harsh and can be misleading because they break covalent bonds. Thus, the present invention sought a method for the study of lipoprotein structure and stability that uses a detergent, which perturbs lipid-protein structures without altering covalent structure.

### BRIEF SUMMARY OF THE INVENTION

The present invention is directed to a method of remodeling lipoproteins. Lipoproteins can be remodeled or the structure of the lipoprotein is altered by perturbation with detergent and/or incorporation of phospholipids into the lipoprotein. In certain embodiments of the present invention, the altered lipoproteins have altered and/or changed and/or enhanced biological activity, for example, activities associated with cholesterol transport. Thus, the method of the present invention can be considered cardioprotective and, thus it can be used to treat a subject suffering from a cardiovascular disease, such as atherosclerosis.

In further embodiments, the composition of the present invention may also be administered in combination with a known standard therapy to treat atherosclerosis, for example, an anti-cholesterol agent. The anti-cholesterol agent can be cholesterol absorption inhibitors, bile acid sequestrants (cholestryramine, cholestipol and colesevalam), nicotinic acid, fibric acids (gemfibrozil, fenofibrate and clofibrate) and HMG-coA reductase inhibitors (lovastatin, pravastatin, simvastatin, fluvastatin, atorvastatin and cerivastatin). Other therapies can include surgery for example providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

An embodiment of the present invention comprises a method of increasing the bioactivity of a lipoprotein and/or the lipoprotein associated-activity comprising the step of administering to a sample a composition having a detergent. After the addition of the detergent, the detergent may be removed by any known method of detergent removal, such as dialysis, ion exchange, gel filtration, detergent-binding agents that can selectively remove the detergent, or the concentration of the detergent is diluted such that it is below the CMC concentration of the detergent.

The sample can be blood, more specifically, the sample is plasma or serum. The sample can be procured from a mammal, more specifically a human. Bioactivity of lipoproteins or the biological activity associated with lipoproteins, can include, for example, increasing cholesterol binding affinity, increasing lecithin:cholesterol acyltransferase (LCAT) activity, increasing cholesterol esterification, increasing lipid metabolism, decreasing hyperlipidemia, and/or decreasing atherosclerosis in a human.

The detergent can effect the activity associated with the total plasma lipoproteins (TLP) in the sample. More specifically, the lipoprotein is a high density lipoprotein (HDL) or a low density lipoprotein (LDL).

In certain embodiments, the detergent is a non-detenaturing detergent. More specifically, the detergent is an anionic detergent or a non-ionic detergent or bile acid or combination thereof. The anionic detergent can be a cholate or a bile acid. More specifically, the cholate detergent is sodium cholate. The amount of the detergent can be from about 0.1 to 100% of its aqueous solubility. More specifically, the amount of detergent is in the range of about 1 times the critical micelle concentration (CMC), about 2 times the CMC, about 3 times the CMC, about 4 times the CMC, about 5 times the CMC, about 6 times the CMC, about 7 times the CMC, about 8 times the CMC, about 9 times the CMC and about 10 times the CMC, wherein the range that is 8 times the critical micelle concentration being optimal.

In further embodiments, the method further comprises administering a phospholipid. The phospholipid is phosphatidylcholine. More specifically, the phospholipid is lecithin. The amount of phospholipid is in the range of about 10 mg/liter of plasma to about 10 g/liter of plasma. More specifically, the amount of phospholipid is about 3 g/liter of plasma. Thus, in certain embodiments, the composition comprises a phospholipid and a detergent, wherein the composition alters the biological activity assoicated with lipoproteins. The ratio of detergent to phospholipid is in the range of about 1:10, 1:5, 1:2, 4:5, 1:1, 1.5:1, 2:1, 3:1, 6:1, 15:1, 20:1, 50:1, 100:1, 200:1 or about 500:1 or any range therebetween. D/PL=0.1, 0.2, 0.5, 0.8, 1.0, 1.5, 2.0, 3.0, 6.0, 15.0, 20.0, 50.0, 100.0, 200.0, and 500.0. In certain embodiments the preferred phospholipid is phosphatidylcholine (PC), thus detergent perturbation is used to enrich or enhance lipoproteins resulting in a PC-enriched lipoprotein.

Another embodiment of the present invention comprises a method of increasing reverse cholesterol transport in a sample comprising the step of administering to the sample a composition comprising a detergent and a phospholipid.

Yet further, embodiment comprises a method of increasing lipid metabolism in a subject suffering from hyperlipidemia comprising the steps of obtaining a blood sample from the subject; treating the blood sample with a detergent and a phospholipid, and administering the treated blood sample to the subject, wherein the treated blood sample increases lipid metabolism and transport in the subject. In certain embodiments, the treated blood sample is dialyzed to remove the detergent prior to administering the treated sample to the subject. The blood sample may also comprise a plasma sample and/or a serum sample.

Thus, in certain embodiments of the present invention a sample, for example a plasma sample, is procurred from a subject suffering from hyperlipidemia. The sample is treated with a composition comprising a detergent and a phospholipid. The composition alters the structure of the lipoproteins in the sample to enhance or increase the activity associated with the lipoproteins, for example cholesterol binding affinity, lecithin:cholesterol acyltransferase (LCAT) activity, increasing cholesterol esterification, increasing removal of the altered lipoprotein and cholesterol from plasma. Next the detergent is removed from the sample and the sample is reinfused into the subject.

Still further, another embodiment comprises a method of regulating the levels of cholesterol in a subject comprising the steps of: i) measuring the levels of cholesterol in a subject, if the levels of cholesterol are above normal, then a treatment sample is obtained from the subject; ii) administering to the treatment sample a composition comprising a detergent and a phospholipid followed by removal of the detergent by dialysis, dilution, ion exchange or size exclusion chromatography, for example; iii) administering the treatment sample of step iii); iv) repeating steps i-iii until the cholesterol level of the subject is at a satisfactory level.

A further embodiment comprises a method of treating a subject suffering from a cardiovascular disease comprising the step of administering to the subject a composition comprising a detergent. The composition further comprises a phospholipid. The composition having either detergent and/or a phospholipid or both can be cardioprotective. The step of administering comprises treating a blood sample with the composition ex vivo or extracorpreal prior to the administering step. The blood sample is autologous, heterologous, and/or homologous.

More specifically, the cardiovascular disease is atherosclerosis. The composition increases the process of reverse cholesterol transport (RCT). RCT is increased by increasing the cholesterolphilicty of a lipoprotein and/or increasing the esterification of cholesterol by lecithin:acyltransferase.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 shows size exclusion chromatography (SEC) of TLP after detergent perturbation. TLP and sodium cholate were combined to give final concentrations of 1.95 mg/mL and 90 mM, respectively and split into three samples. Cholate was removed from one sample by SEC over a column of BioGel P6 DG; cholate was removed from another sample by exhaustive dialysis at 4° C.; the third sample was dilute below the CMC of cholate by the addition of a 9-fold excess of TBS. A control sample in which TBS was substituted for the same volume of cholate was also prepared. SEC profiles of each sample over Superose HR6 are as follows: A) Control TLP; B) TLP+cholate after removal of the cholate by SEC; C) TLP+cholate after removal of the cholate by dialysis; D) TLP+cholate after dilution to 9 mM cholate.

FIGS. 2A-2G shows the kinetics of cholate dialysis. FIG. 2A shows that [³H]Cholate and TLP were mixed to final concentrations of 66 mM and 1.95 mg/mL, respectively and dialyzed for 48 hours during which samples were removed for liquid scintillation counting. A control in which TBS was substituted for the cholate cholate solution was also tested. According to a first order regression analysis of the data, the half times for the disappearance of [³H]cholate were 3.5 and 4.5 hours, respectively for cholate only and cholate+TLP. FIGS. 2A-2G show SEC of TLP (2.0 mg/mL) after mixing with sodium cholate to a final concentration of 90 mM. The dialysis times are as indicated 0 hrs (FIG. 2B), 3 hrs (FIG. 2C), 6 hrs (FIG. 2D), 14 hrs (FIG. 2E), 22 hrs (FIG. 2F) and 47 hrs (FIG. 2G).

FIGS. 3A and 3B show SDS PAGE of Pooled TLP fractions after Detergent perturbation under non reducing (NR) and reducing (R) conditions. The closed curves, labeled F1, F2 and F3 (FIG. 3A), indicate the pooled fractions that were used for compositional analysis and SDS-PAGE (FIG. 3B); Lanes 1 and 2: Standards containing apos A-I, A-II and rA-II. Lanes 3 and 5: Fraction 2. Lanes 4 and 6: Fraction 3. Lanes 3 through 6 were loaded with equal amounts of protein.

FIGS. 4A and 4B show effect of detergent perturbation on the association of Protein (●), phospholipid (◯), and total cholesterol (▪), with lipoprotein fractions isolated by SEC. FIG. 4A shows TLP. FIG. 4B shows TLP after detergent perturbation. Cumulative protein (▴) and phospholipid (Δ) as percentage of total. The curves for protein and phospholipid (as choline) are shifted upward for easier comparison; the vertical scales are the same for all curves in each panel. Initial TLP and cholate concentrations were 2.0 mg/mL and 90 mM, respectively.

FIGS. 5A-5H show cholate dose dependence of TLP SEC profiles. TLP (final concentration=1.95 mg/mL) and various aliquots of 465 mM cholate were mixed at ice temperature and exhaustively dialyzed in a cold room for 48 hours after which each sample was analyzed by SEC over Superose HR6. The initial cholate concentrations are indicated in each panel. FIG. 5A shows 0 mM of cholate. FIG. 5B shows 10 mM of cholate. FIG. 5C shows 15 mM of cholate. FIG. 5D shows 20 mM of cholate. FIG. 5E shows 40 mM of cholate. FIG. 5F shows 90 mM of cholate. FIG. 5G shows 180 mM of cholate. FIG. 5H shows 360 mM of cholate. The elution positions for VLDL, LDL, and HDL are adjacent to the labels in the upper panel.

FIG. 6 shows correlation of apo A-I optical absorbance (280 nm) with initial cholate concentration. The dash line is the first order line of regression for the change in optical absorbance between 0 and 90 mM. The concentrations for the CMC and the plateau of absorbance are indicated by arrows.

FIGS. 7A-7H shows dose dependence of the SEC profile of TLP as a function of its concentration at constant initial cholate concentration (90 mM). The concentrations of TLP vary from 9 to 300% (9%, FIG. 7A; 18%, FIG. 7B; 30%, FIG. 7C; 45%, FIG. 7D; 76%, FIG. 7E; 120%, FIG. 7F; 180%, FIG. 7G; and 300%, FIG. 7H) of the plasma concentration of the donor (1.95 mg/mL TLP-protein). The arrows and adjacent vertical lines locate the elution positions of untreated LDL and HDL.

FIGS. 8A-8F show superose HR6 profiles of TLP after treatment with constant ratio of cholate to TLP, the cholate was removed by dialysis. FIG. 8A shows the chromatographic profile for a sample containing TLP equal to the plasma concentration of the donor (1.95 mg/mL) and 180 mM cholate. The profiles shown in FIGS. 8B-8F are of samples that are identical to that in FIG. 8A but reduced to the percentage of FIG. 8A as shown (67%, FIG. 8B; 44%, FIG. 8C, 28%, FIG. 8D, 19%, FIG. 8E, 11%, FIG. 8F). The dashed vertical lines represent the elution positions of LDL and HDL as shown.

FIGS. 9A-9E show the effect of LDL and HDL concentrations on the detergent perturbation of lipoproteins as assessed by SEC. FIG. 9A shows control LDL+HDL before and after detergent perturbation (DP); FIG. 9B shows detergent perturbation at constant LDL+various HDL concentrations; FIG. 9C shows detergent perurbation at constant HDL+various LDL concentrations. H=HDL; L=LDL. FIG. 9D shows shift in the peak heights of HDL and lipid-poor apo A-I as a function of LDL/HDL ratio. FIG. 9E shows shift in the peak elution voilume of LD as a function of LDL/HDL ratio.

FIGS. 10A and 10B show mean Superose HR6 profiles of TLP from eleven volunteers before (FIG. 10A) and after (FIG. 10B) Detergent perturbation. For Detergent perturbation, the TLP concentration was adjusted to the original plasma TLP concentration by dilution with TBS. The plots show the mean (solid line)±SD (dashed). The change in SEC profile after Detergent perturbation may be seen by referring to the vertical lines that cross the peaks for native LDL and HDL.

FIG. 11 shows a model for the Detergent perturbation remodeling of TLP.

1) formation of a PC/cholate micelle and apo A-I desorption; 2) fusion of PC-poor and apo A-I-poor HDL; 3) with cholate removal, PC returns to LDL and HDL.

FIGS. 12A-12D show the comparison of the lipid metabolism of various rHDL by SR-BI^{+/+} CHO cells. FIG. 12A shows binding; FIG. 12B shows CE uptake; FIG. 12C shows efflux; and FIG. 12D shows competition. rHDL contain apo A-I (which in a color representation may be represented by red, ●▪), apo A-II (which in a color representation may be represented by blue, ●▪), and reduced apo A-II (which in a color representation may be represented by green, ●▪). Control ldlA cells (□); SR-BI+/+ cells (∘).

FIGS. 13A and 13 B show incorporation of POPC into Plasma by DP. Plasma (1-mL), [³H]POPC liposomes (2 mg in 0.2 mL TBS) and 465 mM sodium cholate (0.2 mL) were combined and exhaustively dialyzed at room temperature or 4° C. (Arrow). At the end of dialysis the turbidity of each sample was estimated from the absorbance at 325 nm (%) each sample was centrifuged to sediment uncombined liposomes and the turbidity measured again (%), as shown in FIG. 13A. FIG. 13B shows results of a duplicate procedure conducted with [³H]POPC showing the percent of radioactivity sedimented by centrifugation.

FIGS. 14A-14G show SEC of POPC-enriched TLP. Concentrated TLP (protein=13.2 mg/mL; phospholipid=8.2 mg/mL), 465 mM sodium cholate, TBS, and various amounts of [³H]POPC were combined to give a final concentrations 1.95 mg/mL protein and 66 mM cholate and exhaustively dialyzed at 4° C. FIG. 14A shows 0.00 mg/ml of POPC. FIG. 14B shows 0.18 mg/ml of POPC. FIG. 14C shows 0.36 mg/ml of POPC. FIG. 14D shows 0.72 mg/ml of POPC. FIG. 14E shows 1.08 mg/ml of POPC. FIG. 14F shows 3.23 mg/ml of POPC. FIG. 14G shows 4.93 mg/ml of POPCAliquots (0.2 mL) were analyzed by SEC; analysis is presented as absorbance (280 nm) for protein with (- - - -) and without (-) DP and as radioactivity for [³H]POPC (●-●).

FIGS. 15A-15C show analysis of SEC data of FIG. 14. FIG. 15 A shows percent [³H]POPC radioactivity appearing in void volume. FIG. 15B shows percent of lipoprotein-associated [³H]POPC radioactivity in HDL (●-●) and non-HDL (∘-∘) fractions. FIG. 15C shows total lipoprotein-associated POPC mass associated with HDL (▪-▪) and non-HDL (∘-∘) fractions. Non-HDL and HDL included fractions 3 to 12 and 13 to 23, respectively of FIG. 14.

FIGS. 16A-D show SEC analysis of [³H]cholesterol-labeled TLP as a function of PC content. [³H]cholesterol-labeled TLP was modified by DP in the presence of various amounts of added POPC and analyzed by SEC. TLP (2 mg/mL) was combined with POPC to give final concentrations of 0.0, 0.82, and 1.65 mg/mL (FIGS. 16B, 16C, and 16D, respectively) and enough cholate (465 mM) to give a final concentration of 90 mM and dialyzed. FIG. 16A shows TLP (1.95 mg/mL) without added cholate or POPC. The data were plotted as column effluent absorbance (-) and the radioactivity associated (-) with collected fractions.

FIGS. 17A-17L show SEC analysis of LDL and HDL as a function of PC doses added by DP. Isolated LDL (left) and HDL (right) were modified by DP in the presence of various amounts of added POPC and analyzed by SEC. LDL (0.44 mL, 6.0 mg/mL), cholate (0.80 mL; 465 mM) and POPC (0 (FIG. 17A) 50 (FIG. 17B), 150 (FIG. 17C), 250 (FIG. 17D), and 375 μL (FIG. 17E); 26.3 mM (FIG. 17F)) were combined with enough TBS to give a final volume of 4 mL and an LDL-protein concentration of 0.65 mg/mL. HDL (0.50 mL; 10.4 mg/mL), cholate (0.80 mL; 465 mM) and POPC (0 (FIG. 17G) 50 (FIG. 17H), 150 (FIG. 17I), 250 (FIG. 17J), and 375 μL (FIG. 17K); 26.3 mM (FIG. 17L)) were combined with enough TBS to give a final volume of 4 mL with an HDL-protein concentration of 1.30 mg/mL.

FIGS. 18A and 18B show the effect of POPC Enrichment of TLP, LDL and HDL on Cholesterophilicity. FIG. 18A shows cholesterophilicity of TLP was based on KP the partitioning of cholesterol between CDX and TLP as a function of POPC added to the TLP by DP. According to a first order linear regression analysis, Kₚ=45[POPC]+0.019; r²=0.82. (●) TLP+various doses of POPC after DP; (∘) TLP without added POPC or DP. Data are presented as the means±SD. FIG. 18C shows that the TLP-PC content is linear with the amount of added POPC. FIG. 18B shows cholesterophilicity of LDL and HDL after detergent mediated enrichment of isolated lipoproteins with POPC. Data are plotted as a function of the amount of LDL-(∘) or HDL-associated (●) phospholipid. Triplicate values are also shown for untreated native LDL (□) and HDL (Δ). Box denotes cloudy samples with material appearing in the void volume of SEC.

### DETAILED DESCRIPTION OF THE INVENTION

I. Definitions

Unless defined otherwise, technical and scientific terms used herein have the same meaning as commonly understood by one of ordinary skill in the art to which this invention belongs. For purposes of the present invention, the following terms are defined below.

As used herein, the use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

As used herein, the term “allogeneic” refers to cells being genetically different, but deriving from the same species.

As used herein, the term “autologous” refers to tissue, cells or stem cells that are derived from the same subject's body.

The term “atherosclerosis” as used herein includes a form of arteriosclerosis characterized by a combination of changes in the intima of arteries, such changes include, but are not limited to accumulation of lipids, complex carbohydrates, blood and blood products, fibrous tissue and calcium deposits. Yet further, atherosclerotic plaques can be characterized into at least two areas. One type is characterized by prominent proliferation of cells with small accumulations of lipids. The second type consists mostly of intracellular and extracellular lipid accumulation and a small amount of cellular proliferation.

As used herein, the term “cardiovascular disease or disorder” refers to disease and disorders related to the cardiovascular or circulatory system. Cardiovascular disease and/or disorders include, but are not limited to, diseases and/or disorders of the pericardium (i.e., pericardium), heart valves (i.e., incompetent valves, stenosed valves, Rheumatic heart disease, mitral valve prolapse, aortic regurgitation), myocardium (coronary artery disease, myocardial infarction, heart failure, ischemic heart disease, angina) blood vessels (i.e., arteriosclerosis, aneurysm) or veins (i.e., varicose veins, hemorrhoids). Yet further, one skill in the art recognizes that cardiovascular diseases and/or disorders can result from congenital defects, genetic defects, environmental influences (i.e., dietary influences, lifestyle, stress, etc.), and other defects or influences.

The term “cholesterol” as used herein refers to the monohydric alcohol form, which is a white, powdery substance that is found in all animal cells and in animal-based foods (not in plants). Cholesterol is an essential nutrient necessary for many functions, including the following: repairing cell membranes, manufacturing vitamin D on the skin's surface, production of hormones, such as estrogen and testosterone, and possibly helping cell connections in the brain that are important for learning and memory.

The term “chylomicron” as used herein refers to the largest in size and lowest in density of the triglyceride carrying lipoproteins.

The term “critical micelle concentration” or CMC” as used herein refers to the concentration of a surfactant and/or detergent in a solution and/or composition at which the molecules begin to form aggregates called micelles while the concentration of surfactant and/or detergent in solution remains constant.

The term “dyslipidemia” refers to any altered amount of any or all of the lipids or lipoproteins in the blood. Dyslipidemia can be hyperlipidemia, hyperlipoproteinemia, hypercholesterolemia, hypertriglyceridemia, HDL deficiency, and ApoA-I deficiency. Disorders associated with dyslipidemia include cardiovascular disease, coronary artery disease, atherosclerosis or restenosis. It is to be understood that the term dyslipidemia refers to the disorders, cardiovascular disease, coronary artery disease, atherosclerosis or restenosis. In preferred embodiments, the treatment is for a human disorder.

As used herein, the term “ex vivo” refers to “outside” the body. One of skill in the art is aware that ex vivo and in vitro can be used interchangeably.

As used herein, the term “extracorpreal” refers to “outside” the body.

As used herein, the term “heterologous” refers to tissue, cells or stem cells that are derived from the different species.

As used herein, the term “homologous” refers to tissue, cells or stem cells that are derived from the same species.

The term “high-density lipoprotein” or “HDL” as used herein is the smallest and most dense type of cholesterol-carrying lipoprotein and is often referred to as the “good” cholesterol.

The term “intermediate density lipoprotein” or “IDL” as used herein refers to a triglyceride-carrying lipoprotein.

The term “lipoproteins” as used herein are protein spheres that transport cholesterol, triglyceride, or other lipid molecules through the bloodstream. Lipoproteins are categorized into five types according to size and density. They can be further defined by whether they carry cholesterol [the two smaller lipoproteins (HDL and LDL)] or triglycerides [the three largest lipoproteins (IDL, VLDL, and chylomicrons)]. Still further, lipoprotein also includes LP(a), apolipoproteins (such as apoAI), or other proteins which complex with lipids.

The term “lipid” as used herein refers to the building blocks of any of the fats or fatty substances found in animals and plants, which are characterized by their insolubility in water and solubility in fat solvents such as alcohol, ether and chloroform. Lipids include fats (e.g., esters of fatty acids and glycerol); lipoids (e.g., phospholipids, cerebrosides, waxes) and sterols (e.g., cholesterol).

The term “low density lipoprotein” or “LDL” as used herein is a type of cholesterol-carrying lipoprotein which is often called the “bad” cholesterol.

The term “plasma” as used herein refers to the liquid part of the blood and lymphatic fluid, which makes up about half of its volume. Plasma is devoid of cells and, unlike serum, has not clotted. Blood plasma contains antibodies and other proteins. It is taken from donors and made into medications for a variety of blood-related conditions. Some blood plasma is also used in non-medical products.

The term “preventing” as used herein refers to minimizing, reducing or suppressing the risk of developing a disease state or parameters relating to the disease state or progression or other abnormal or deleterious conditions.

The term “statin” as used herein includes compounds that are HMG-CoA reductase inhibitors, for example, but not limited to simvastatin (Zocor®) and atorvastatin (Lipitor®). Thus, as used herein the terms “statin” and “HMG-CoA reductase inhibitor” are interchangeable.

The term “satisfactory level” as used herein refers to the level of cholesterol in a subject. One of skill in the art realizes that a subject having an above normal level of cholesterol can be treated to lower or reduce the levels of cholesterol to a satisfactory level. This satisfactory level may be a normal level of cholesterol or it may be slightly higher. This level can be determined by those of skill in the art and may be subject to other factors or indicators that the subject possess.

As used herein, the term “subject” may encompass any vertebrate including but not limited to humans, mammals, reptiles, amphibians and fish. However, advantageously, the subject is a mammal such as a human, or other mammals such as a domesticated mammal, i.e., dog, cat, horse, and the like, or production mammal, i.e., cow, sheep, pig, and the like. In a specific preferred embodiment, the methods of the present invention are employed to treat a human subject. In more preferred embodiments, the subject has signs or indicators of cardiovascular disease, more specifically, atherosclerosis. These signs or indicators include, for example, the development of cholesterol plaques in the arteries and calcification, the extent of which can be determined by Sudan IV staining, or the development of foam cells in an artery or arterial spasm. Atherosclerosis also is characterized by a narrowing of the arteries detected by, for example, coronary angioplasty, ultrasound and ultrafast CT. In further embodiments, the subject is at risk of developing a cardiovascular disease. Thus, the subject may or may not be cognizant of their disease state or potential disease state and may or may not be aware that they are need of treatment (therapeutic treatment or prophylactic treatment).

The term “therapeutically effective amount” as used herein refers to an amount that results in an improvement or remediation of the symptoms of the disease or condition.

The term “treating” and “treatment” as used herein refers to administering to a subject a therapeutically effective amount of a the composition so that the subject has an improvement in the disease. The improvement is any improvement or remediation of the symptoms. The improvement is an observable or measurable improvement. Thus, one of skill in the art realizes that a treatment may improve the disease condition, but may not be a complete cure for the disease.

The term “total cholesterol” as used herein refers to the sum of three kinds of lipids: high-density lipoprotein (HDL), low-density lipoprotein (LDL), and triglycerides. Levels of serum total cholesterol of >200 mg/dl are levels that are an indicating risk factor for atherosclerosis and cardiovascular disease.

The term “triglycerides” as used herein are composed of fatty acid molecules and are the basic chemicals contained in fats in both animals and plants.

The term “very low density lipoprotein” or “VLDL” as used herein refers to a triglyceride carrying lipoprotein.

II. Reverse Cholesterol Transport

RCT comprises three steps: cellular cholesterol efflux from peripheral tissues to various early forms of HDL; remodeling of early forms of HDL in the plasma compartment; and uptake of lipid in mature forms of HDL by hepatic receptors.

A. Cholesterol Efflux

Cholesterol efflux from peripheral tissue occurs through at least four mechanisms (Yancey et al., 1995). One is mediated by the interaction of an apolipoprotein such as apo A-I or apo A-II, the two major HDL apos, with the ABCA1 transporter, which triggers the unidirectional release of cholesterol and phospholipid (PL), mostly phosphatidylcholine (PC) forming early forms of HDL (Hara and Yokoyama, 1991; Bielicki et al., 1992; Yancey et al., 1995). Variants of ABCA1 are associated with Tangier disease and some forms of hypo-lipoproteinemia, in which cellular transfer of cholesterol to lipid-free apos is impaired (Okuhira et al., 2004; Francis et al., 1995; Yancey et al., 2003). Two other transporters, ABCG1 or ABCG4, are also important in RCT. ABCG1 and ABCG4 mediates cholesterol efflux to HDL (Jian et al., 1997; Huttunen et al., 1988), but not to lipid-poor apoA-I (Wang et al., 2004; Nakamura et al., 2004). These two transporters, which are highly expressed in macrophages, could mediate cholesterol efflux from macrophage foam cells to the major HDL fractions, thereby providing the mechanistic link that may underlies the epidemiological association of elevated HDL-C and reduced CVD risk. A third mechanism is the reversible spontaneous cholesterol desorption from the plasma membrane into the surrounding aqueous phase where it associates with early forms of HDL. Spontaneous cholesterol efflux is driven by a gradient in cholesterol concentration from high (donor) to low (acceptor); high relative levels of sphingomyelin in the acceptor increase efflux and greatly reduce the reverse transfer (Phillips et al., 1987; Phillips et al., 1988; Lund-Katz et al., 1988). Finally, SR-B1, a mediator of selective hepatic uptake of HDL-CE, -TG, and PL,20 also mediates the cellular efflux of cholesterol to HDL. Cholesterol efflux via SR-BI has an absolute, requirement for PL in the acceptor. This requirement is dose- and PL species-dependent; respective PL enrichment and depletion of the acceptor increase and decrease efflux by this receptor, and if PC in the acceptor is replaced by a more cholesterophilic PL such as sphingomyelin, efflux is enhanced (Yancey et al., 2000 and Jian b et al., 1997).

Given the requirement of PC-containing species for spontaneous, and ABCG1/4- and SR-BI-mediated efflux, the present invention provides for the addition of a highly cholesterophilic PC to HDL that elicits the greatest increase in cellular cholesterol efflux.

B. Remodeling of Lipoproteins in the Plasma

A general structure of a lipoprotein includes, a core consisting of a droplet of triacylglycerols and/or cholesteryl esters, a surface monolayer of phospholipid, unesterified cholesterol and specific proteins (apolipoproteins, e.g., apoprotein B-100 in low density lipoprotein). As is known and understood in the art, lipoproteins differ in their content of proteins and lipids. (See Methods of Enzymology Volumnes 128 and 129, Havel et al., Liporptoeins and Lipid Transport, each of which is incorporated herein by reference) They are classified based on their density: chylomicron (largest; lowest in density due to high lipid/protein ratio; highest in triacylglycerols as % of weight); VLDL (very low density lipoprotein; 2nd highest in triacylglycerols as % of weight); IDL (intermediate density lipoprotein); LDL (low density lipoprotein, highest in cholesteryl esters as % of weight); and HDL (high density lipoprotein, highest in density due to high protein/lipid ratio).

Early forms of HDL are modified by plasma factors and continue to receive additional cholesterol and PLs from other cellular sites and plasma lipoproteins. The most important plasma factor in this pathway, LCAT, catalyzes transfer of the SN-2 acyl chain of PC to cholesterol thereby forming CE. Microscopically, this step converts cholesterol from a form that freely diffuses among membranes and lipoproteins to a form that is absolutely nonexchangeable in the absence of plasma factors. Macroscopically, early forms of HDL are converted from discs with free cholesterol intercalated among the PLs, to spheroids, which characterize mature forms of HDL in which CE is confined to a molten core. With additional acquisitions of cholesterol and PC and subsequent esterification, HDL grows to its larger mature form, HDL (Jian B et al., 1997). LCAT activity is stimulated by apo A-I and varies according to the structure and environment of the PC that donates the acyl chain to cholesterol; in the absence of cholesterol, LCAT has phospholipase A2 activity, which requires enzyme binding to a PC surface (Aron et al., 1978). From this it is inferred that LCAT binds to the PC component of lipoproteins and that addition of PC to lipoproteins not only increases the number of acyl donors but also provides more PC surface for LCAT binding, an obligatory component of its activity. LCAT activity is highest for PCs in a fluid environment and maximum reactivity is observed with chain lengths of 14 and 16 carbons for saturated PCs, and 16 and 18 carbons for monounsaturated species, which have the highest reactivity of the SN-1-monounsaturated species (Pownall et al., 1985). Thus, the present invention remodels lipoproteins, for example HDL, LDL, by adding PC. Still further, the addition of PC to plasma lipoproteins enhances cholesterol esterification by LCAT by providing more PC surface for binding and more acyl donors.

Two plasma lipid transfer activities, PLTP and CETP, also affect lipoprotein composition and structure. PLTP mediates PL transfer between all lipoproteins, with its main activity being against HDL. PLTP converts HDL to larger species by fusion with the concomitant release of apo A-I (Lusa et al., 1996; Rao et al., 1997), an activity that is emulated with great fidelity by DTR of HDL. Although the physiological role of PLTP is difficult to assess with certainty, it may be important in remodeling HDL. The effects of CETP are more profound; physiologically, CETP exchanges HDL-CE for VLDL-TG. In hypertriglyceridemic patients, this process is the main contributor to low plasma HDL-C levels (Pownall et al., 1999). Because CETP lowers plasma HDL-C, it is thought to be an atherogenic activity. However, studies of patients with CETP deficiency do not support this hypothesis suggesting that in addition to HDL levels, flux of cholesterol via RCT is an important determinant of CVD risk (Zhong et al., 1996).

C. Hepatic Uptake of Lipid in Mature Forms of HDL

The terminal step in RCT is hepatic uptake via SR-BI, a receptor that binds disparate lipoprotein ligands, including HDL and rHDL comprising apo A-I and PC, and delivers the lipidic components, CE, PL, and TG, to cells through a selective lipid uptake mechansim wherein HDL-proteins are largely excluded from net uptake (Rigotti et al., 2003). SR-BI has a higher affinity for the large, CE-rich, HDL than for the relatively lipid-poor pre-β-HDL (Liadaki et al., 2000; de Beer et al., 2001) or lipid-free apoA-I (Liadaki et al., de Beer et al., 2001, Pilon et al., 2000). According to a current model, lipid uptake occurs in two-steps, lipoprotein binding and lipid transfer (de Beer et al., 2001; Williams et al., 2000, Thuahnai et al, 2003) so that cellular assays of this component must measure CE internalization and not just cell surface binding. Association of SR-BI with HDL is a function of the apo A-I conformation (de Beer et al., 2001) and like many other activities of apo A-I, is antagonized by apo A-II even though binding is actually increased (de Beer et al., 2001; Pilon et al., 2000). Binding and cross-linking studies show SR-BI interacts with multiple amphipathic α-helical motifs in apo A-I (Thuahnai et al, 2003). Although HDL from apo A-I and apo A-I+/+ mice bind SR-BI with similar affinities, selective uptake of CE from apo A-I+/+ HDL is 3-fold higher than from apo A-I HDL (Temel et al., 2002). Thus, apo A-I is important if not essential to the recognition of HDL by SR-BI.

It follows that for improved cholesterol efflux from peripheral sites to be atheroprotective, uptake of the mature forms of HDL by hepatic HDL receptors must also be enhanced. Thus, the present invention utilizes modified lipoproteins that are made PC-rich by DP (detergent perturbation) and converted to more mature CE-rich forms by LCAT and other plasma factors and exhibit increased CE transfer to cells via SR-BI.

III. Detergent

Detergents are amphiphilic substances that are monomeric at low concentrations but at higher concentrations self associate to form noncovalently associated oligomeric structures known as micelles (Tanford 1980). Detergents have been used to reconstitute the proteins and activities of cell membranes (Racker) and human plasma lipoproteins, particularly HDL; e.g., sodium cholate “catalyzes” the association of apo A-I with lipids giving rHDL (Pownall et al, 1982; Matz and Jonas, 1982a) as well as cholesterol-containing rHDL (Pownall et al, 1982; Matz and Jonas, 1982b). Herein, the present invention utilizes detergent perturbation. Detergent perturbation (DR) revealed differences in the structures and stabilities of lipoproteins and emulated the activity of human plasma phospholipid transfer protein. Thus, the present invention comprises a composition having a detergent that is used to alter the structure of the lipoprotein thereby altering the activity associated with the lipoprotein. Activity associated with lipoproteins or the biological activity of lipoproteins, as used herein, refer to, for example, cholesterol binding affinity, lecithin:cholesterol acyltransferase (LCAT) activity, increasing cholesterol esterification, increases lipid metabolism, decreases hyperlipidemia, and/or decreases atherosclerosis in a human. Lipoproteins as used herein can refer to total plasma lipoprotein (TLP). In certain embodiments, lipoproteins can refer to individual or specific lipoproteins, for example, but not limited to HDL, LDL, IDL, VLDL, and chylomicrons. More specifically, the the lipoprotein is a high density lipoprotein (HDL) or a low density lipoprotein (LDL).

The ionic character of the polar head group forms the basis for broad classification of detergents; they may be ionic (charged, either anionic or cationic), nonionic (uncharged) or zwitterionic (having both positively and negatively charged groups but with a net charge of zero). The non-ionic detergents disrupt non-covalent polar bonds and are less effective in dissociating protein complexes. The non-ionic detergents tend to form large micelles. Ionic detergents disrupts polar bonds and hydrophobic bonds. The ionic detergents can be anionic (SDS, cholate, deoxycholate), cationic (alkyltriemthylammonium salts), zwitterionic (CHAP, zwittergent).

Detergents at low concentration in aqueous solution form a monolayer at the air-liquid interface. At higher concentrations, detergent monomers aggregate into structures called micelles. Micelles are a thermodynamically stable colloidal aggregate of detergent monomers wherein the nonpolar ends are sequestered inward, avoiding exposure to water, and the polar ends are oriented outward in contact with the water. The number of detergent monomers per micelle (aggregation number) and the range of detergent concentration above which micelles form (called the critical micelle concentration, CMC) are properties specific to each particular detergent. Detergent properties can also be affected by experimental conditions for example, concentration, temperature, buffer pH and ionic strength, and the presence of various additives.

Detergents can be denaturing or non-denaturing with respect to protein structure. Denaturing detergents can be anionic such as sodium dodecyl sulfate (SDS) or cationic such as ethyl trimethyl ammonium bromide. These detergents totally disrupt membranes and denature proteins by breaking protein:protein interaction. Non-denaturing detergents can be divided into nonionic detergents such as Triton® X-100, anionic detergents, such as bile salts and cholate, and zwitterionic detergents such as CHAPS.

Non-denaturing agents do not bind to native conformations nor do they have a cooperative binding mechanism. These detergents have rigid and bulky apolar moieties that do not penetrate into water soluble proteins. They bind to the hydrophobic parts of proteins. Triton® X100 and other polyoxyethylene nonanionic detergents are inefficient in breaking protein-protein interaction and can cause artifactual aggregations of protein. These detergents will, however, disrupt protein-lipid interactions but are much gentler and capable of maintaining the native form and functional capabilities of the proteins. It is envisioned that non-denaturing detergents are used in the present invention.

One of skill in the art is aware that there are several methods to remove detergents from a sample. Dialysis works well with detergents that exist as micelles that coexist with monomers. Dialysis is somewhat ineffective with detergents that readily aggregate to form micelles with very low critical micelle concentrations because they micelles are too large to pass through dialysis membrane and the monomers occur at a concentration that permits only a very slow rate of esecape. Ion exchange chromatography can be utilized to circumvent this problem. The disrupted protein solution is applied to an ion exchange chromatography column and the column is then washed with buffer minus detergent. The detergent will be removed as a result of the equilibration of the buffer with the detergent solution. Alternatively the protein solution may be passed through a density gradient. As the protein sediments through the gradients the detergent will come off due to a difference in chemical potential that favors dissociation into monomers, a process that is supported by a concentration gradient.

Often a single detergent is not versatile enough for the solubilization and analysis of the milieu of proteins found in a cell. The proteins can be solubilized in one detergent and then placed in another suitable detergent for protein analysis. The protein-detergent micelles formed in the first step should separate from pure detergent micelles. When these are added to an excess of the detergent for analysis, the protein is found in micelles with both detergents. Separation of the detergent-protein micelles can be accomplished with ion exchange or gel filtration chromatography, dialysis or buoyant density type separations.

In certain embodiments, the detergent concentration of the present compositions may comprise about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or any range derivable therein, of the present compositions by weight or volume. Thus, it is contemplated that the detergents of the present compositions may comprise any of the detergents or other components in any combination or percentage range.

More specifically, the amount of the detergent can be from about 0.1 to 100% of its aqueous solubility, with a range that is 8 times the critical micelle concentration being optimal. Critical micelle concentration (CMC) is the concentration of an amphiphilic component (detergent) in solution at which the formation of aggregates (micelles, round rods, lamellar structures etc.) in the solution is initiated.

Thus, of interest to the present invention is determining the amount detergent that results in molecular aggregates called “micelles” which are defined as colloidal aggregates spontaneously formed by amphiphilic compounds in water above a critical solute concentration, the critical micellar concentration (CMC), and at solution temperatures above the critical micellar temperature (CMT). The molecules constituting the micelles are in rapid dynamic equilibrium with the unassociated molecules. The increase in the concentration above the CMC usually leads to an increase in the number of micelles without any change in micellar size; however, in certain cases with phospholipid mixed micelles, the spherical micelles enlarge into rod-shaped micelles (Carey et al., 1972; Hjelm, Jr. et al., 1992). The CMC is strongly temperature dependent, and at a given concentration the monomer to micelle transition occurs gradually over a broad temperature range (Almgren et al., 1995). An increase in the temperature leads to an increase in the number of aggregates, while the hydrodynamic radius remains constant (Nivaggioli et al., 1995); Alexandridis et al., 1995). In general the increase in temperature leads to an increase in hydrophobic interactions and the water dielectric constant is reduced augmenting the ionic repulsion forces. There are many ways to determine the CMC of an amphiphilic compound (surface tension measurements, solubilization of water insoluble dye, or a fluorescent probe, conductivity measurements, light scattering, and the like). Thus, one of skill can use any of the known methods to determine the CMC, some of which are further defined in US Patent Application No. US20050025819, U.S. Pat. No. 6,322,810 and International Publication WO0000815, each of which are incorporated herein by reference.

A. Anionic Detergents

More preferable, the detergents used in the present invention are anionic, such as sodium cholate and bile salts. Anionic detergents are detergents, often an alkyl sulfonate or alkaryl sulfonate, which has a negatively charged functional group. Anionic detergents can be either denaturing or non-denaturing detergents.

Examples of anionic detergents include, but are not limited to chenodeoxycholic acid, chenodeoxycholic acid sodium salt, cholic acid, ox or sheep bile, dehydrocholic acid, deoxycholic acid, deoxycholic acid, deoxycholic acid methyl ester, digitonin, digitoxigenin, N,N-dimethyldodecylamine N-oxide, docusate sodium salt, glycochenodeoxycholic acid sodium salt, glycocholic acid hydrate, glycocholic acid sodium salt hydrate, glycodeoxycholic acid monohydrate, glycodeoxycholic acid sodium salt, glycolithocholic acid 3-sulfate disodium salt, glycolithocholic acid ethyl ester, N-lauroylsarcosine sodium salt, N-lauroylsarcosine solution, lithium dodecyl sulfate, lugol solution, niaproof 4,1-Octanesulfonic acid sodium salt, sodium 1-butanesulfonate, sodium 1-decanesulfonate, sodium 1-heptanesulfonate anhydrous, sodium 1-nonanesulfonate, sodium 1-propanesulfonate monohydrate, sodium 2-bromoethanesulfonate, sodium cholate hydrate, sodium choleate, sodium deoxycholate, sodium deoxycholate monohydrate, sodium dodecyl sulfate, sodium hexanesulfonate anhydrous, sodium octyl sulfate, sodium pentanesulfonate anhydrous, sodium taurocholate, taurochenodeoxycholic acid sodium salt, taurodeoxycholic acid sodium salt monohydrate, taurohyodeoxycholic acid sodium salt hydrate, taurolithocholic acid 3-sulfate disodium salt, tauroursodeoxycholic acid sodium salt, approx. 90%, Trizma® dodecyl sulfate, and ursodeoxycholic acid.

More specifically, the anionic detergents used in the present invention are non-denaturing agents, which include for example, sodium cholate and salts thereof, or bile salts.

B. Triton® X-Detergents:

This family of detergents (Triton® X-100, X114 and NP-40) have the same basic characteristics but are different in their specific hydrophobic-hydrophilic nature. All of these heterogeneous detergents have a branched 8-carbon chain attached to an aromatic ring. This portion of the molecule contributes most of the hydrophobic nature of the detergent. Triton® X detergents are used to solubilize membrane proteins under non-denaturing conditions. The choice of detergent to solubilize proteins will depend on the hydrophobic nature of the protein to be solubilized. Hydrophobic proteins require hydrophobic detergents to effectively solubilize them.

Triton® X-100 and NP-40 are very similar in structure and hydrophobicity and are interchangeable in most applications including cell lysis, delipidation protein dissociation and membrane protein and lipid solubilization. Generally 2 mg detergent is used to solubilize 1 mg membrane protein or 10 mg detergent/1 mg of lipid membrane. Triton® X-114 is useful for separating hydrophobic from hydrophilic proteins.

C. Brij® Detergents

These are similar in structure to Triton® X detergents in that they have varying lengths of polyoxyethylene chains attached to a hydrophobic chain. However, unlike Triton® X detergents, the Brij® detergents do not have an aromatic ring and the length of the carbon chains can vary. The Brij® detergents are difficult to remove from solution using dialysis but may be removed by detergent removing gels. Brij®58 is most similar to Triton® X100 in its hydrophobic/hydrophilic characteristics. Brij®-35 is a commonly used detergent in HPLC applications.

D. Tween® Detergents

The Tween® detergents are non-denaturing, non-ionic detergents. They are polyoxyethylene sorbitan esters of fatty acids. Tween® 20 and Tween® 80 detergents are used as blocking agents in biochemical applications and are usually added to protein solutions to prevent nonspecific binding to hydrophobic materials such as plastics or nitrocellulose. They have been used as blocking agents in ELISA and blotting applications. Generally, these detergents are used at concentrations of 0.01-1.0% to prevent nonspecific binding to hydrophobic materials.

Tween® 20 and other nonionic detergents have been shown to remove some proteins from the surface of nitrocellulose. Tween® 80 has been used to solubilize membrane proteins, present nonspecific binding of protein to multiwell plastic tissue culture plates and to reduce nonspecific binding by serum proteins and biotinylated protein A to polystyrene plates in ELISA.

The difference between these detergents is the length of the fatty acid chain. Tween® 80 is derived from oleic acid with a C 18 chain while Tween® 20 is derived from lauric acid with a C 12 chain. The longer fatty acid chain makes the Tween® 80 detergent less hydrophilic than Tween® 20 detergent. Both detergents are very soluble in water.

The Tween® detergents are difficult to remove from solution by dialysis, but Tween® 20 can be removed by detergent removing gels. The polyoxyethylene chain found in these detergents makes them subject to oxidation (peroxide formation) as is true with the Triton® X and Brij® series detergents.

E. Dialyzable Nonionic Detergents

η-Octyl-β-D-glucoside (octylglucopyranoside) and β-Octyl-β-D-thioglucoside (octylthioglucopyranoside, OTG) are non-denaturing non-ionic detergents which are easily dialyzed from solution. These detergents are useful for solubilizing membrane proteins and have low UV absorbances at 280 nm. Octylglucoside has a high CMC of 23-25 mM and has been used at concentrations of 1.1-1.2% to solubilize membrane proteins.

Octylthioglucoside was first synthesized to offer an alternative to octylglucoside. Octylglucoside is expensive to manufacture and there are some inherent problems in biological systems because it can be hydrolyzed by β-glucosidase.

F. Zwitterionic Detergents

The zwitterionic detergent, CHAPS, is a sulfobetaine derivative of cholic acid. This zwitterionic detergent is useful for membrane protein solubilization when protein activity is important. This detergent is useful over a wide range of pH (pH 2-12) and is easily removed from solution by dialysis due to high CMCs (8-10 mM). This detergent has low absorbances at 280 nm making it useful when protein monitoring at this wavelength is necessary. CHAPS is compatible with the BCA Protein Assay and can be removed from solution by detergent removing gel. Proteins can be iodinated in the presence of CHAPS.

CHAPS has been successfully used to solubilize intrinsic membrane proteins and receptors and maintain the functional capability of the protein. When cytochrome P-450 is solubilized in either Triton® X-100 or sodium cholate aggregates are formed.

G. Surfactants

In addition to the above referenced detergents, biological detergents or surfactants can be used. Generally, surfactants are water-soluble surface-active agents comprised of a hydrophobic portion, usually a long alkyl chain, attached to hydrophilic or water solubility enhancing functional groups.

Groups of surfactants that can be used in the present invention include, but are not limited to alkyl benzene sulfonates (ABS), linear Alkyl benzene sulfonates (LAS), alkyl phenoxy polyethoxy ethanols (alcohol ethoxylates). Other surfactants that can be used in the present invention are short chain phospholipids, for examle, but not limited to phosphatidylcholines containing acyl chains with 4 (e.g., diacetyl) to 24 (e.g., dilauroyl) carbons that form micelles or mixed micelles.

IV. Phospholipids

In one embodiment of the present invention comprises a method of increasing the activity associated with a lipoprotein comprising the step of administering to a sample a composition having a detergent, wherein the composition further comprises a phospholipid, thereby resulting in an altered lipoprotein structure thereby altering the activities associated with the lipoprotein. Thus, detergent perturbation of a lipoprotein can result in a lipoprotein being enriched and/or enhanced with a phospholipid due to the incorporation of phospholipids into the lipoprotein. This altered lipoprotein or phospholipid enhanced or enriched lipoprotein alters the activity associated with the lipoprotein. Activities associated with lipoproteins or the biological activity of lipoproteins, as used herein, refer to cholesterol binding affinity, lecithin:cholesterol acyltransferase (LCAT) activity, increasing cholesterol esterification, increases lipid metabolism, decreases hyperlipidemia, and/or decreases atherosclerosis in a human. The lipoprotein is a high density lipoprotein (HDL) or a low density lipoprotein (LDL).

A phospholipid generally comprises either glycerol or an sphingosine moiety, an ionic phosphate group to produce an amphipathic compound, and one or more fatty acids. Types of phospholipids include, for example, phosphoglycerides, wherein a phosphate group is linked to the first carbon of glycerol of a diglyceride, and sphingophospholipids (e.g., sphingomyelin), wherein a phosphate group is esterified to a sphingosine amino alcohol. For example, in some embodiments, the lipid molecule is dilaurylphosphatidylcholine, a phospholipid which includes two fully saturated fatty acid moieties.

A phospholipid may, of course, comprise further chemical groups, such as for example, an alcohol attached to the phosphate group. One of ordinary skill in the art would be familiar with the broad class of agents known as phospholipids, and the chemical groups which may be attached to phospholipids.

In certain embodiments, the phospholipid is phoshatidylcholine, which is also known to be a major constituent of cell membranes. Phosphatidylcholine is also known as 1,2-diacyl-:ussn:ue-glycero-3-phosphocholine, PtdCho and lecithin. The phospholipid may also be 1-palmitoyl-2-oleoyl (PO) PC.

Commercially available lecithin may comprise a mixture of neutral and polar lipids. Phosphatidylcholine, which is a polar lipid, can be present in commercial lecithin in concentrations of 10 to 100%. Thus, in the present invention, lecithin can comprise at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100% or any range derivable therebetween of phoshaptidylcholine.

Natural soursces of lecithins containing phosphatidylcholine are produced from vegetable, animal and microbial sources, but mainly from vegetable sources. Soybean, sunflower and rapeseed are the major plant sources of commercial lecithin. Soybean is the most common source.

A phosphoplipid is classified as a biological lipid. Thus, the present invention is not limited to phospholipids. It is contemplated that other lipids can also be used in the present invention as long as they result in altered lipoprotein activity as described above. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long chain aliphatic hydrocarbons and their derivatives. A lipid may be naturally occurring or synthetic (i.e., designed or produced by an orgnaims, more specifically a human). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

The phospholipid or lipid concentration of the present compositions may comprise about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or any range derivable therein, of the present compositions by weight or volume. Thus, it is contemplated that the lipid or lipids of the present compositions may comprise any of the phospohlipids, lipid types or other components in any combination or percentage range.

V. Treatment and/or Prophylaxis of Cardiovascular Disease

In accordance with the present invention, the composition of the present invention is administered to a subject who has experienced or is at risk of developing cardiovascular disease. Risk factors include, but are not limited to elevated levels of cholesterol. One of skill in the art can determine the patients who would potentially benefit from a therapeutic agent that would reduce circulating levels of total cholesterol or triglycerides. One of skill in the art can determine the therapeutically effective amount of the composition to be administered to a subject based upon several considerations, such as local effects, pharmacodynamics, absorption, metabolism, method of delivery, age, weight, disease severity and response to the therapy.

Cardiovascular diseases and/or disorders include, but are not limited to, diseases and/or disorders of the pericardium, heart valves (e.g., incompetent valves, stenosed valves, Rheumatic heart disease, mitral valve prolapse, aortic regurgitation), myocardium (e.g., coronary artery disease, myocardial infarction, heart failure, ischemic heart disease, angina) blood vessels (e.g., hypertension, arteriosclerosis, aneurysm) or veins (e.g., varicose veins, hemorrhoids). In specific embodiments, the cardiovascular disease is atherosclerosis.

The present invention is directed to a method of remodeling lipoproteins. Lipoproteins can be remodeled or the structure of the lipoprotein is altered by the perturbation with detergent and/or incorporation of phospholipids into the lipoprotein, thereby resulting in a phospholipid rich lipoprotein. The altered lipoproteins have altered and/or changed and/or enhanced biological activity, for example, activities associated with cholesterol transport. Thus, the method of the present invention can be considered cardioprotective and, thus it can be used to treat a subject suffering from a cardiovascular disease, such as atherosclerosis.

One embodiment of the present invention comprises a method of increasing the activity associated with a lipoprotein comprising the step of administering to a sample a composition having a detergent that increases the activity associated of the lipoprotein. Activities associated with lipoproteins or the biological activity of lipoproteins, as used herein, refer to, for example, cholesterol binding affinity, lecithin:cholesterol acyltransferase (LCAT) activity, increasing cholesterol esterification, increases lipid metabolism, decreases hyperlipidemia, and/or decreases atherosclerosis in a human. Lipoproteins in the present invention include total plasma lipropteins. More specifically, the lipoprotein is a high density lipoprotein (HDL) or a low density lipoprotein (LDL).

The sample of the present invention can include a blood sample, a plasma sample or a serum sample. The sample is a mammalian sample, more specifically a human sample. In certain embodiments, the sample is autologous, heterologous, and/or homologous.

The detergent is a non-denaturing detergent, more specifically, an anionic detergent, a non-ionic detergent or a bile acid. An effective amount of the detergent in the composition that may be administered includes a dose of about 0.1 mM to about 400 mM. More specifically, doses of the detergent to be administered are from about 0.1 m to about 10 mM; about 1 mM to about 5 mM; about 5 mM to about 10 mM; about 10 mM to about 15 mM; about 15 mM to about 20 mM; about 20 mM to about 30 mM; about 30 mM to about 40 mM; about 40 mM to about 50 mM; about 50 mM to about 60 mM; about 60 mM to about 70 mM; about 70 mM to about 80 mM; about 80 mM to about 90 mM; about 90 mM to about 100 mM, about 100 mM to about 110 mM, about 110 mM to about 120 mM, about 120 mM to about 130 mM, about 130 mM to about 140 mM, about 140 mM to about 150 mM, about 150 mM to about 160 mM, about 160 mM to about 170 mM, about 170 mM to about 180 mM, about 180 mM to about 190 mM, about 190 mM to about 200 mM, about 200 mM to about 250 mM, about 250 mM to about 300 mM, about 300 mM to about 350 mM, and about 350 mM to about 400 mM. Of course about all of these amounts are exemplary, and any amount in-between these points is also expected to be of use in the invention.

The amount of detergent that is administrered is in the range of about zero to about saturation. As known by those of skill in the art, saturation is the point at which a solution of a substance can dissolve no more of that substance. Alternatively, the amount of the detergent can be from about 0.1 to 100% of its aqueous solubility. More specifically, the amount of detergent that is administered is in the range of about 1 times the CMC, about 2 times the CMC, about 3 times the CMC, about 4 times the CMC, about 5 times the CMC, about 6 times the CMC, about 7 times the CMC, about 8 times the CMC, about 9 times the CMC and about 10 times the CMC, wherein the range that is 8 times the critical micelle concentration being optimal. As discussed previously, there are many ways to determine the CMC of an amphiphilic compound, for example, but not limited to surface tension measurements, solubilization of water insoluble dye, or a fluorescent probe, conductivity measurements, light scattering, and the like. Thus, one of skill can use any of the known methods to determine the CMC, some of which are further defined in US Patent Application No. US20050025819, U.S. Pat. No. 6,322,810 and International Publication WO0000815, each of which are incorporated herein by reference. Once the CMC is determined, then that amount is increased to achieve the range that is about 1 times the CMC, about 2 times the CMC, about 3 times the CMC, about 4 times the CMC, about 5 times the CMC, about 6 times the CMC, about 7 times the CMC, about 8 times the CMC, about 9 times the CMC and about 10 times the CMC and so forth.

The composition can further comprise a phospholipid. The phospholipid is phosphatidylcholine (PC). The amount of phospholipid is in the range of about 10 mg/liter of plasma to about 10 g/liter of plasma. More specifically, the amount of phospholipid is about 3 g/liter of plasma.

Thus, addition of phospholipids, more specifically PC to plasma is likely to enhance cholesterol efflux, LCAT activity, and selective uptake of HDL-CE. All three of these steps may be optimized by choosing a PC that is the highly cholesterophilic and a good acyl donor for cholesterol esterification. Given the reported differences in the cholesterophilicity of various PC species, efflux would be best supported by acceptors containing I-stearoyl-2-oleoy- or 1-palmitoyl-2-oleoy PC, both of which are highly cholesterophilic. LCAT activity is highest for PCs in a fluid environment and maximum reactivity is observed with acyl chain lengths of 14 and 16 carbons for saturated PCs, and 16 and 18 carbons for monounsaturated species, which have the highest reactivity of the SN-1-monounsaturated species. Although disaturated PCs are good LCAT substrates, the short chained analogs (C₁₄) are rapidly cleared via spontaneous transfer and the long chained analogs form solid lipid domains that do not provide an optimal environment for LCAT activity. This leaves 1-stearoyl-2-oleoy- or 1-palmitoyl-2-oleoy PC, both of which are good LCAT substrates and highly cholesterophilic. Thus, lipoprotein therapy (for example HDL and/or LDL therapy) that includes a PC that is both a good cholesterol acceptor, i.e., highly cholesterophilic, and a good LCAT substrate should will ensure production of a mature form of HDL is CE-rich.

In certain embodiments of the present invention the composition comprises a detergent and a phospholipid. The ratio of detergent to phospholipid is in the range of about 1:10, 1:5, 1:2, 4:5, 1:1, 1.5:1, 2:1, 3:1, 6:1, 15:1, 20:1, 50:1, 100:1, 200:1 or about 500:1 or any range therebetween. In certain embodiments the preferred phospholipid is phosphatidylcholine (PC), thus detergent perturbation is used to enrich or enhance lipoproteins resulting in a PC-enriched lipoprotein.

The composition of the present invention is added to the sample (blood, plasma, and/or serum). Once the sample is treated with the composition, the detergent is removed via any standard detergent removal methods, for example dialysis. In addition to removal, dilution of the detergent can be used to dilute the concentration of the detergent to one that is below the CMC of the detergent. Once the detergent is removed or diluted, the treated sample can be administered to a subject or reinfused into the subject.

Another embodiment includes treating a human subject with an elevated level of circulating total cholesterol according to the then medically established guidelines. It is contemplated that the composition of the present invention reduces or attenuates the levels of circulating total cholesterol.

In a preferred embodiment of the present invention, the composition is administered in an effective amount to decrease, reduce, inhibit or abrogate cardiovascular disease. Thus, a subject is administered a therapeutically effective amount of a composition so that the subject has an improvement in the parameters relating to cardiovascular disease including circulating levels of total cholesterol, HDL, LDL, VLDL, and trigylcerides. More specifically, the subject is administered a treated sample, in which a sample, for example a plasma sample, has been treated with the composition of the present invention (i.e., detergent and/or phospholipid or a combination thereof). This treated sample is then reinfused or administered to the subject resulting in an improvement in the parameters relating to cardiovascular disease including circulating levels of total cholesterol, HDL, LDL, VLDL, and trigylcerides.

The effective amount or “effective amounts” of the composition to be used are those amounts effective to produce beneficial results, particularly with respect to cardiovascular disease treatment, in the recipient animal or patient. Such amounts may be initially determined by reviewing the published literature, by conducting in vitro tests or by conducting metabolic studies in healthy experimental animals. Before use in a clinical setting, it may be beneficial to conduct confirmatory studies in an animal model, preferably a widely accepted animal model of the particular disease to be treated. Preferred animal models for use in certain embodiments are rodent models, which are preferred because they are economical to use and, particularly, because the results gained are widely accepted as predictive of clinical value. Thus, as used herein, the term effective amount can refer to the amount of the detergent and/or phospholipid and/or a combination thereof that is used to treat a sample, for example a plasma sample. Yet further, the term effective amount can refer to the actual amount of treated plasma or blood sample that is infused or administered to a subject.

As is well known in the art, a specific dose level of active compounds of the composition such as the detergent and/or phospholipid and/or combinations thereof for any particular patient depends upon a variety of factors including the activity of the specific compound employed, the age, body weight, general health, sex, diet, time of administration, route of administration, rate of excretion, drug combination, and the severity of the particular disease undergoing therapy. The person responsible for administration will determine the appropriate dose for the individual subject. Moreover, for human administration, preparations should meet sterility, pyrogenicity, general safety and purity standards as required by FDA Office of Biologics standards.

A therapeutically effective amount of the composition of the present invention as a treatment varies depending upon the host treated and the particular mode of administration. In one embodiment of the invention the dose range of the composition thereof will be about 0.5 mg/kg body weight to about 500 mg/kg body weight. The term “body weight” is applicable when an animal is being treated. When isolated samples are being treated, “body weight” as used herein should read to mean “total sample body weight”. The term “total body weight” may be used to apply to both isolated sample and animal treatment. All concentrations and treatment levels are expressed as “body weight” or simply “kg” in this application are also considered to cover the analogous “total cell body weight” and “total body weight” concentrations. However, those of skill will recognize the utility of a variety of dosage range, for example, 1 mg/kg body weight to 450 mg/kg body weight, 2 mg/kg body weight to 400 mg/kg body weight, 3 mg/kg body weight to 350 mg/kg body weight, 4 mg/kg body weight to 300 mg/kg body weight, 5 mg/kg body weight to 250 mg/kg body weight, 6 mg/kg body weight to 200 mg/kg body weight, 7 mg/kg body weight to 150 mg/kg body weight, 8 mg/kg body weight to 100 mg/kg body weight, or 9 mg/kg body weight to 50 mg/kg body weight. Further, those of skill will recognize that a variety of different dosage levels will be of use, for example, 1 mg/kg, 2 mg/kg, 3 mg/kg, 4 mg/kg, 5 mg/kg, 7.5 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 120 mg/kg, 140 mg/kg, 150 mg/kg, 160 mg/kg, 180 mg/kg, 200 mg/kg, 225 mg/kg, 250 mg/kg, 275 mg/kg, 300 mg/kg, 325 mg/kg, 350 mg/kg, 375 mg/kg, 400 mg/kg, 450 mg/kg, 500 mg/kg, 550 mg/kg, 600 mg/kg, 700 mg/kg, 750 mg/kg, 800 mg/kg, 900 mg/kg, 1000 mg/kg, 1250 mg/kg, 1500 mg/kg, 1750 mg/kg, 2000 mg/kg, 2500 mg/kg, and/or 3000 mg/kg. Of course, all of these dosages are exemplary, and any dosage in-between these points is also expected to be of use in the invention. Any of the above dosage ranges or dosage levels may be employed for composition of the present invention.

Treatments may include various “unit doses.” Unit dose is defined as containing a predetermined quantity of the composition calculated to produce the desired responses in association with its administration, i.e., the appropriate route and treatment regimen. The quantity to be administered, and the particular route and formulation, are within the skill of those in the clinical arts. Also of import is the subject to be treated, in particular, the state of the subject and the protection desired. A unit dose need not be administered as a single injection but may comprise continuous infusion over a set period of time.

The improvement is any observable or measurable improvement. Thus, one of skill in the art realizes that a treatment may improve the patient condition, but may not be a complete cure of the disease. In certain aspects, the composition is administered in an effective amount to decrease, reduce, inhibit or abrogate excess amounts of cholesterol levels in circulation. A subject requires treatment for cholesterol levels based upon any of the following situations: LDL of 160 mg/ml or greater; LDL of 130-159 mg/ml and also have two or more cardiovascular risk factors; LDL of 100 mg/ml or greater in subjects with coronary heart disease (CHD); triglycerides of 200 mg/dl or higher; total cholesterol of 240 mg/dl or higher or HDL of less than 40 mg/dl. Thus, after administration of the composition, if any of the above conditions improve, then the amount of the composition is considered an effective amount to decrease, reduce, inhibit or abrogate cholesterol levels in the circulation.

Yet further, another embodiment is a method of preventing a cardiovascular disease in a subject at risk for developing a cardiovascular disease comprising the step of administering to the subject a composition of the present invention in an amount sufficient to result in prophylaxis of the cardiovascular disease in the subject. In preferred embodiments, the cardiovascular disease is atherosclerosis. It is envisioned that the composition not only possess therapeutic benefits for those subjects suffering from cardiovascular diseases, but also possess prophylactic properties for those subjects at risk for developing cardiovascular disease. A subject at risk may or may not be cognizant of their disease state or potential disease state and may or may not be aware that they are need of treatment.

Prophylactic treatment can be administered to those subjects at risk for developing atherosclerosis. One risk factor is an atherogenic lipoprotein profile. For example, a ratio of serum cholesterol to high density lipoproteins of above 5:1 indicates a higher than average risk of developing atherosclerosis. Other factors indicating increased risk for atherosclerosis include a serum cholesterol level of above 240 mg/dl; a high density lipoprotein level below about 35 mg/dl; and a low density lipoprotein level above about 160 mg/dl.

The present invention also comprises a method of increasing lipid metabolism in a subject suffering from hyperlipidemia comprising the steps of obtaining a blood sample from the subject; treating the blood sample with a detergent and a phospholipid to the blood sample; administering the treated blood sample to the subject, wherein the treated blood sample increases lipid metabolism in the subject.

Still further, another embodiment comprises a method of regulating the levels of cholesterol in a subject comprising the steps of: i) measuring the levels of cholesterol in a subject, if the levels of cholesterol are above normal, a treatment sample is obtained from the subject; ii) treating the sample with a composition comprising a detergent and a phospholipid; iii) administering the sample in step iii); iv) repeating steps i-iii until the cholesterol level of the subject is at a satisfactory level. A satisfactory level may be a level at or near the normal level of cholesterol as determined by those of skill in the art or it may be higher or lower than normal depending upon other factors, such as other risk for cardiovascular disease and the levels of LDL, HDL, and/or triglycerides or a combination thereof.

A further embodiment comprises a method of treating a subject suffering from a cardiovascular disease comprising the step of administering to the subject a composition comprising a detergent. The composition further comprises a phospholipid. The step of administering comprises treating a blood sample with the composition ex vivo prior to the administering step. The blood sample is autologous, heterologous, and/or homologous.

More specifically, the cardiovascular disease is atherosclerosis. The composition increases the process of reverse cholesterol transport (RCT). RCT is increased by increasing the cholesterolphilicty of a lipoprotein and/or increasing the activity of lecithin:cholesterol acyltransferase activity

VI. Combination Treatments

In order to increase the effectiveness of the composition, it may be desirable to combine these compositions and methods of the invention with a known agent effective in the treatment or prevention of cardiovascular disease or disorder, for example known agents to treat or prevent atherosclerosis. In some embodiments, it is contemplated that a conventional therapy or agent, including but not limited to, a pharmacological therapeutic agent, a surgical therapeutic agent (e.g., a surgical procedure) or a combination thereof, may be combined with the composition of the present invention.

The composition of the present invention may precede, be co-current with and/or follow the other agent(s) by intervals ranging from minutes to weeks. In embodiments where the composition of the present invention, and other agent(s) are applied separately to a cell, tissue or organism, one would generally ensure that a significant period of time did not expire between the time of each delivery, such that the composition and agent(s) would still be able to exert an advantageously combined effect on the cell, tissue or organism.

Various combination regimens of the composition and one or more agents are employed. One of skill in the art is aware that the composition of the present invention and agents can be administered in any order or combination. In other aspects, one or more agents may be administered substantially simultaneously, or within about minutes to hours to days to weeks and any range derivable therein, prior to and/or after administering the composition.

Administration of the composition to a sample or organism may follow general protocols for the administration of cardiovascular therapeutics, taking into account the toxicity, if any. It is expected that the treatment cycles would be repeated as necessary. In particular embodiments, it is contemplated that various additional agents may be applied in any combination with the present invention.

A. Pharmacological Therapeutic Agents

Pharmacological therapeutic agents and methods of administration, dosages, etc. are well known to those of skill in the art (see for example, the “Physicians Desk Reference”, Goodman & Gilman's “The Pharmacological Basis of Therapeutics”, “Remington's Pharmaceutical Sciences”, and “The Merck Index, Eleventh Edition”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Non-limiting examples of a pharmacological therapeutic agent that may be used in the present invention include an antihyperlipoproteinemic agent, an antiarteriosclerotic agent, an anti-cholesterol agent, an anti-inflammatory agent, an antithrombotic/fibrinolytic agent, a blood coagulant, an antiarrhythmic agent, an antihypertensive agent, or a vasopressor. In certain aspects of the present invention, anti-cholesterolemic agents are used in combination with the composition of the present invention. Anti-cholesterol agents include but are not limited to HMG-CoA Reductase inhibitors, cholesterol absorption inhibitors, bile acid sequestrants, nicotinic acid and derivatives thereof, fibric acid and derivatives thereof. HMG-CoA Reductase inhibitors include statins, for example, but not limited to atorvastatin calcium (Lipitor®), cerivastatin sodium (Baycol®), fluvastatin sodium (Lescol®), lovastatin (Advicor®), pravastatin sodium (Pravachol®), and simvastatin (Zocor®). Agents known to reduce the absorption of ingested cholesterol include, for example, Zetia®. Bile acid sequestrants include, but are not limited to cholestryramine, cholestipol and colesevalam. Other anti-cholesterol agents include fibric acids and derivatives thereof (e.g., gemfibrozil, fenofibrate and clofibrate); nicotinic acids and derivatives thereof (e.g., nician, lovastatin) and agents that extend the release of nicotinic acid, for example niaspan. Other agents can include for example, chelation therapy, fibrates, omega-3 fattay acid, and ezitimibe.

B. Surgical Therapeutic Agents

In certain aspects, a therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

VII. Formulations and Routes for Administration

Where clinical applications are contemplated, it will be necessary to prepare pharmaceutical compositions of the present invention thereof, or any additional therapeutic agent disclosed herein in a form appropriate for the intended application. Generally, this will entail preparing compositions that are essentially free of pyrogens, as well as other impurities that could be harmful to humans or animals.

Aqueous compositions of the present invention in an effective amount may be dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route. The drugs and agents also may be administered parenterally or intraperitoneally. The term “parenteral” is generally used to refer to drugs given intravenously, intramuscularly, or subcutaneously.

Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The therapeutic compositions of the present invention may be administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH, exact concentration of the various components, and the pharmaceutical composition are adjusted according to the well known parameters. Suitable excipients for formulation with the present composition include croscarmellose sodium, hydroxypropyl methylcellulose, iron oxides synthetic), magnesium stearate, microcrystalline cellulose, polyethylene glycol 400, polysorbate 80, povidone, silicon dioxide, titanium dioxide, and water (purified).

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

VIII. Therapeutic Kits

Any of the compositions described herein may be comprised in a kit. Therapeutic kits of the present invention are kits comprising at least a detergent. Further kits may comprise a detergent in combination with a phospholipid. Such kits will generally contain, in suitable container means, a pharmaceutically acceptable formulation of detergent and/or phospholipid. The kit may have a single container means, and/or it may have distinct container means for each compound.

When the components of the kit are provided in one and/or more liquid solutions, the liquid solution is an aqueous solution, with a sterile aqueous solution being particularly preferred. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the detergent and/or phospholipid is suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Irrespective of the number and/or type of containers, the kits of the invention may also comprise, and/or be packaged with, an instrument for assisting with the injection/administration and/or placement of the composition within the body of an animal. Such an instrument may be a syringe, pipette, forceps, and/or any such medically approved delivery vehicle.

IX. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Isolation of TLP

Plasma for lipoprotein isolation was from a donor Center. Except where noted, all experiments were conducted with the total plasma lipoprotein (TLP) from one normolipidemic volunteer. The plasma lipids of the single donor at the time of collection were total cholesterol=184 mg/dL, triglycerides=48 mg/dL, HDL-C=62 mg/dL, and calculated (Friedewald et al, 1972) LDL-C=112 mg/dL. TLP was isolated and concentrated by flotation at d=1.21 g/mL; the concentrated stock TLP-protein and -choline lipid concentrations were 12.2 and 8.2 mg/mL, respectively. LDL and HDL were isolated by sequential flotation at d=1.019, 1.063, and 1.21 g/mL or by preparative size exclusion chromatography (SEC) over Superose HR6. Experiments were conducted over a time interval during which the SEC profile of the TLP on Superose HR6 was unchanged. Tris-buffered saline (TBS; 100 mM NaCl, 10 mM Tris HCl, 0.01% azide, 0.01% EDTA, pH=7.4) was used throughout. Key findings were confirmed using TLP from the plasma of ten other volunteers.

### EXAMPLE 2

### Lipoprotein Composition

Protein was determined according to a modified Lowry method (Markwell et al., 1978). The lipid compositions of native DTR-modified lipoproteins were determined by analysis for cholesterol, CE, TG, PL, and protein using commercial kits and standards supplied by the vendors (Wako Chemicals USA, Inc. Richmond, Va.). Apolipoprotein composition was determined by SDS-PAGE using 18% Tris-Glycine Ready Gels (BioRad). Protein bands were visualized with Pierce GelCode Blue stain reagent or by immunoblotting using a mouse anti human apo A-I monoclonal antibody (Chemicon MAB011-A/13) followed by an HRP chicken anti-mouse IgG antisera and an HRP goat anti human apo A-II antibody (Biodesign International, Saco, M A). Destained gels were photographed with the Kodak Electrophoresis Documentation and Analysis System 290.

### EXAMPLE 3

### Gradient Gel Electrophoresis (GGE)

GGE was performed with 4-15% Tris Glycine Ready Gels (Biorad) according to a method described by Blanche et al (1976) using commercial standards. After staining (0.1% Coomasie Blue R250), gels were destained gels, and photographed with a Kodak Electrophoresis Documentation and Analysis System (EDAS) 290.

### EXAMPLE 4

### Agarose Gel Electrophoresis

Agarose gel electrophoresis was conducted in 90 mM Tris, 80 mM Borate, 3 mM EDTA, pH 8.2 (BUF) using a Gibco-BRL Life Tech Horizon 58 horizontal electrophoresis apparatus. Samples (˜5-10 μg protein/20 μL) in 40% sucrose, 0.05% bromophenol blue, in BUF were transferred to 0.72% Gibco-BRL Life Tech ultrapure agarose. Samples were electrophoresed at 90 volts for 2 h at 4° C. Gels were stained with Pierce GelCode Blue stain reagent 1-2 hr, destained overnight in deionized water, and recorded by photography.

### EXAMPLE 5

### Preparation of DP-Modified Lipoproteins

DP-modified lipoproteins were prepared by mixing reagents in test tubes on ice followed by dialysis (Spectra/Por, r=7.3 mm, molecular weight cutoff for retention ˜6,000-8,000) for 48 hours vs. a 1000-fold excess of TBS at 4° C. with a change of buffer at 24 hours. TLP and 465 mM sodium cholate were mixed to achieve final concentrations of 90 mM cholate and original plasma TLP concentration, nominally 2 mg/mL protein; the concentration of added POPC, which was premixed with sodium cholate. At the end of dialysis, the DTR-modified lipoproteins were separated by SEC on an Amersham-Pharmacia ÄKTA chromatography system equipped with a pair of Superose 6 columns in tandem; 0.5 mL fractions were collected and the peak fractions pooled and analyzed as described below. Typically, each sample was filtered (0.2 μm), 0.2 mL was injected into the chromatography system using a 0.2 mL sample loop, and eluted with TBS. The column effluent was monitored by absorbance at 280 nm. For larger quantities, a 0.5 mL sample loop was used and individual fractions from multiple runs were pooled.

### EXAMPLE 6

### Lipoprotein Analysis After Detergent Perturbation

The effects of detergent perturbation on lipoprotein compositions and SEC profiles were determined by mixing sodium cholate with TLP on ice and transferring the samples to dialysis sacks. Unless otherwise indicated, all samples were twice dialyzed for 24 hours against a >1000 fold excess of TBS at 4 C and then analyzed by SEC using an Amersham-Pharmacia ÄKTA chromatography system equipped with two Superose HR6 columns in tandem. The elution volumes for HDL, LDL and VLDL were determined by chromatography of lipoprotein standards isolated by sequential flotation at d=1.006, 1.063, and 1.21 g/mL (Shumaker and Puppione, 1986). Typically, a sample was filtered (0.2 m), injected into the chromatograph using a 0.2 mL sample loop, and eluted with TBS. The column effluent was monitored by absorbance at 280 nm. For preparative chromatography in which the eluant was collected for further analysis, a 0.5 mL sample loop was used; individual fractions or pooled fractions from multiple runs were analyzed for protein according to Lowry as modified by Markwell et al (1978) and for cholesterol, triglyceride, and PC, using commercial kits (Wako Chemicals USA, Inc. Richmond, Va.). Apolipoprotein composition was determined by SDS PAGE using 4-15% gradient or 18% Tris-Glycine Ready gels (BioRad). Bands were visualized with Pierce GelCode Blue stain reagent, destained, and recorded with the Kodak Electrophoresis Documentation and Analysis System (EDAS) 290.

### EXAMPLE 7

### Effects of Detergent Perturbation on TLP

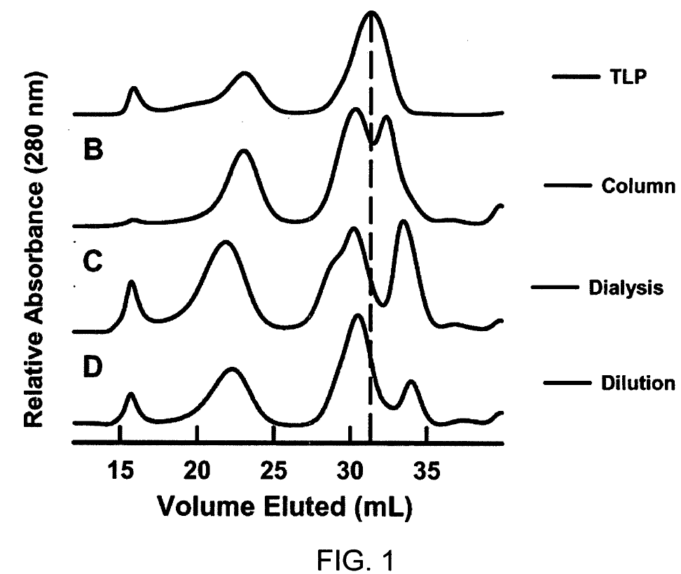

The SEC elution profile of untreated TLP contained prominent peaks that were identified as VLDL, LDL and HDL by comparison with authentic lipoproteins isolated by sequential flotation (FIG. 1). Two other samples of TLP were treated with cholate, which was removed by SEC over BioGel P6 DG or dialysis, the two most common methods used for detergent-mediated formation of rHDL (Pownall et al, 1982; Matz and Jonas, 1982a). Detergent perturbation of TLP by P6-SEC had little effect on the elution positions of VLDL and LDL whereas the peak for HDL was apparently split into two poorly resolved peaks (FIG. 1B). The effects of detergent perturbation by dialysis were more profound (FIG. 1C). The peak for LDL was shifted to a smaller elution volume, which corresponds to a shift to a higher molecular weight species, and HDL eluted as two almost resolved peaks. The effect of dialysis and SEC could be due to total removal of detergent or to the reduction of the detergent concentration below its CMC (15 mM) where it loses its solubilizing power. This was tested by mixing TLP and detergent to the same initial concentrations that were used for SEC and dialysis, and rapidly diluting the mixture to 9 mM cholate. Dilution of the detergent-TLP mixture altered the elution profile of both LDL and HDL (FIG. 1D), an effect that was similar though not identical to that of dialysis (FIG. 1C). Given its experimental simplicity and its more profound effects on the lipoprotein profiles of TLP, the remaining studies of detergent perturbation were conducted using dialysis to remove the detergent.

### EXAMPLE 8

### Dialysis Kinetics

The kinetics of cholate dialysis with and without TLP were determined by dialyzing 66 mM cholate containing [³H]cholate at 4 C, and collecting aliquots at various time intervals. Radioactivity associated with cholate was determined by liquid scintillation counting. TLP and sodium cholate were mixed in in 2.23 mL TBS to a final concentration of 1.95 mg/mL and 90 mM, respectively by the addition of 20% sodium cholate containing [3H]cholate as a tracer. After brief vortexing, a 50 L sample was removed and the remainder was transferred to a dialysis sack. At various time intervals 50 or 100 L aliquots of the retentate were removed and counted.

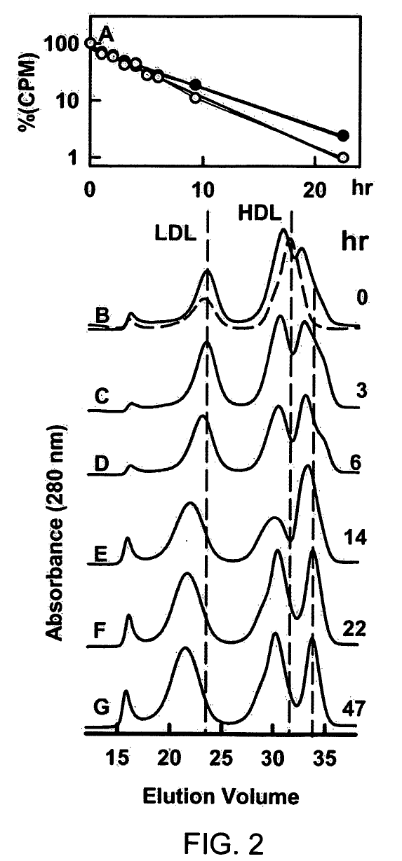

The rate of [³H]cholate escape from a dialysis sack was determined (FIG. 2A, Insert). According to a first order regression analysis of the data, the half times for the disappearance of [³H]cholate were 3.5 and 4.5 hours, respectively for cholate only and cholate+TLP. According to the measured rate constant for dialysis, <0.01% (<9 M) of the original cholate remained inside the dialysis sack after 48 hours.

The kinetics of detergent perturbation of lipoprotein structure were followed by SEC. Cholate and TLP were combined to respective final concentrations of 90 mM and 2.0 mg/mL and an aliquot was analyzed by SEC. The remainder of the sample was dialyzed as described above and aliquots of the retentate were removed at various times and analyzed by SEC. SEC of TLP after addition of 90 mM cholate but before dialysis was begun shows the peak for HDL split into two poorly resolved components and the LDL peak unchanged. With increasing dialysis time, the split in the HDL peak increases and by 22 hours is well resolved and little changed by additional dialysis to 47 hours. Between 6 and 14 hours, the peak for LDL shifts to an earlier elution time with little additional change up to 47 hours. On the basis of these results, subsequent detergent perturbation studies were conducted by dialysis for two days with a change of dialysis buffer after 24 hours at a minimum sample to buffer ratio of 1000.

### EXAMPLE 9

### SDS PAGE of Lipoproteins After Detergent Perturbation

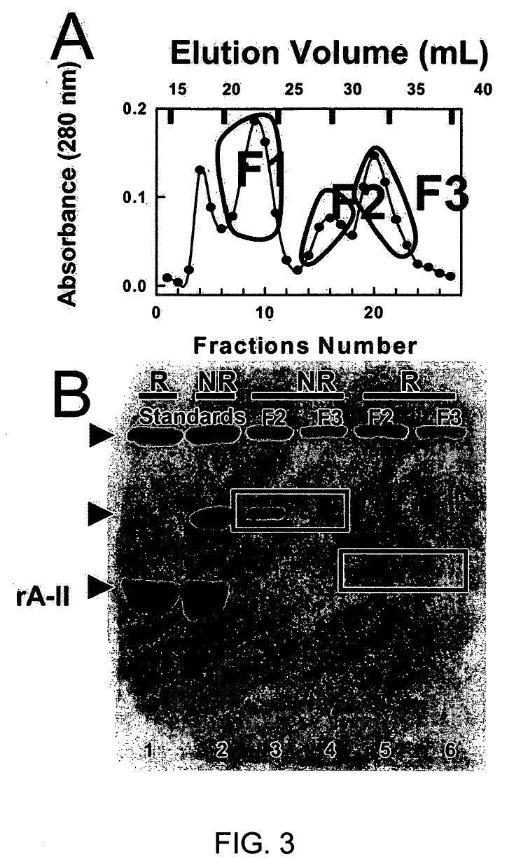

Following detergent perturbation, three fractions were collected for SDS-PAGE analysis under reducing and non reducing conditions (FIG. 3). Analysis of pooled Fraction 1 (F1) by SDS PAGE revealed the presence of one protein, apo B-100. Proteins in pooled Fraction 2 and 3 (F2 and F3) were identified by comparison with authentic samples of apos A-I and A-II (FIG. 3, Lanes 1 and 2, respectively). SDS PAGE of Fraction 2 under non reducing conditions revealed two bands with mobilities that coincided with those of apos A-I and A-II. Under reducing conditions, the band coinciding with that of apo A-II disappeared and a new lower molecular weight band appeared; this further confirmed the identity of apo A-II, which splits from a disulfide-linked dimeric protein into monomeric species under reducing conditions. In contrast, Fraction 3, which on Superose HR6 SEC eluted later than native HDL was composed almost exclusively of one protein, apo A-I, with a barely visible trace of apo A-II (FIG. 3, Lanes 4 and 6). Based on a calibration curve obtained from the elution volumes of standard proteins and the peak HDL fractions before and after detergent perturbation, particle weights of ˜170,000 and 230,000, respectively were calculated; these data were corroborated by non denaturing gradient gel electrophoresis. Detergent perturbation produced a similar shift in the elution position of LDL; the absence of suitable standards precluded a reasonable estimate of the particle weight on the basis of SEC or non denaturing gradient gel electrophoresis. However, according to cryoelectron microscopy the detergent perturbed LDL particles were large and heterogeneous.

### EXAMPLE 10

### Lipoprotein Composition After Detergent Perturbation

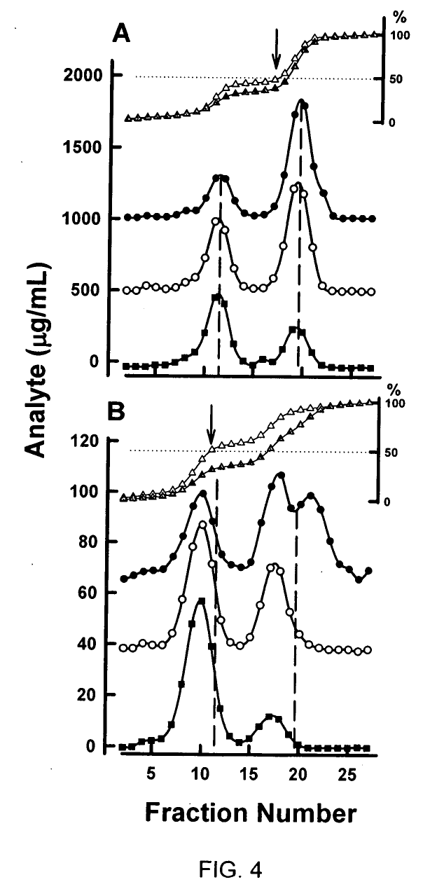

The effluent from SEC before and after detergent perturbation was analyzed for protein, total cholesterol, and phospholipid. These data (FIG. 4) revealed that detergent perturbation also shifts the distribution of TLP-lipids. As expected, before detergent perturbation LDL- and HDL-proteins co-elute with total cholesterol and phospholipids (FIG. 4A). After detergent perturbation, the shifted peaks for LDL and HDL protein co-elute with those for total cholesterol and phospholipid (FIG. 4B). However, the late eluting peak corresponding to Fraction 3 in FIG. 3 A, appears to contain little or no cholesterol or phospholipid, its peak elution volume coinciding with that of isolated apo A-I (0.36 mg/mL).

Analysis of the peak tubes revealed major compositional changes (Table 1). As expected, with the release of apo A-I from HDL, the relative amounts of the lipid components increase; this effect is greatest for phospholipid which increases significantly but not as much as would be predicted on the basis of the release of 50% of the apo A-I, suggesting that some of the phospholipid transfers to other lipoprotein fractions. This can be seen by comparing the cumulative phospholipid (PL) and protein (Pro) content of each fraction across the entire SEC profile before and after detergent perturbation. As shown in the upper curve of FIG. 4A, 50% of the phospholipid in TLP appears in fractions 1 through 18; there is a similar distribution of protein. In contrast, after detergent perturbation, 50% of the phospholipid appears in fractions 1 through 11 whereas the protein is split nearly evenly between fractions 1 through 16 and 17 to 28 (FIG. 4B; upper curve). Thus, detergent perturbation shifts phospholipid but not protein from HDL to the apo B-100-containing lipoproteins.

TABLE 1
Compositions of Isolated TLP Fractions
before and after Detergent Perturbation

| | Control TLP | | TLP + Detergent Perturbation | |
|---|---|---|---|---|
| Analyte | LDL | HDL | LDL | HDL |
| PL | 18.5 ± 0.4 | 27.6 ± 1.5 | 21.1 ± 0.1 | 32.4 ± 0.35 |
| FC | 6.0 ± 0.7 | 1.8 ± 0.4 | 4.8 ± 1.3 | 3.1 ± 0.7 |
| CE | 50.7 ± 4.2 | 16.7 ± 3 | 41.1 ± 2.5 | 15.2 ± 0.2 |
| TG | 5.2 ± 1.2 | 1.7 ± 0.9 | 6.6 ± 0.6 | 5.7 ± 1.1 |
| Protein | 19.6 ± 0.8 | 52.2 ± 0.9 | 26.4 ± 2.9 | 43.6 ± 0.8 |

The fractions pooled for control LDL and HDL and for detergent perturbed LDL and HDL were, respectively, 10-12, 19-20, 9-10, and 19-20 (FIG. 4).

### EXAMPLE 11

### Cholate Dose Dependence of Detergent Perturbation

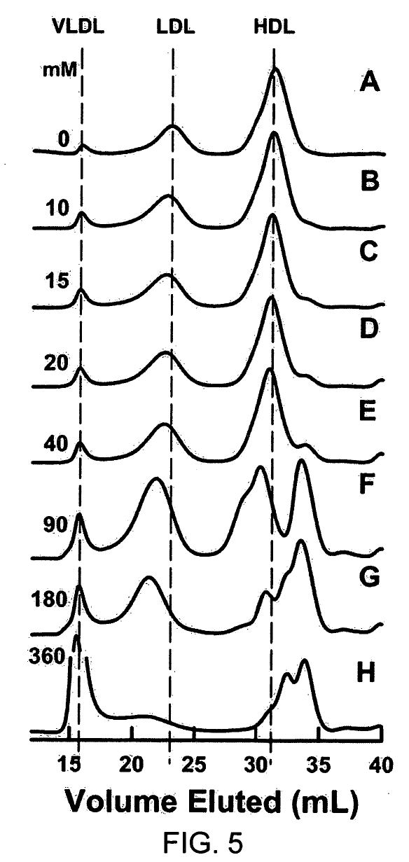

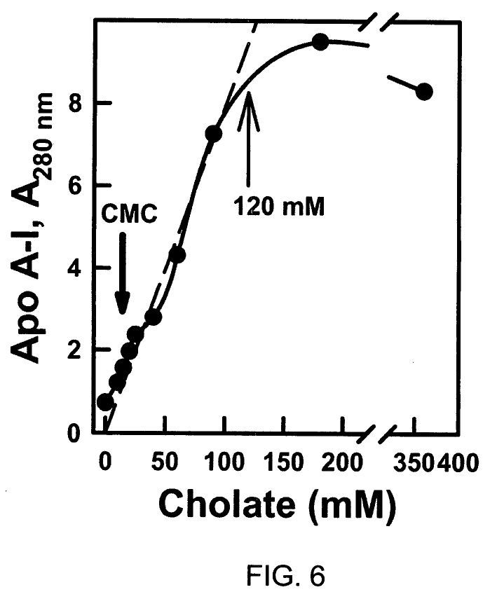

TLP at a final concentration of 1.95 mg/mL was mixed with sufficient cholate to give final concentrations of 0, 5, 10, 15, 25, 40, 60, 90, 120, and 360 mM. The cholate was removed by dialysis, and aliquots of each sample were analyzed by Superose HR6 chromatography in which the relative protein concentrations were estimated from the absorbance at 280 nm (FIG. 5). Between 0 and 25 mM cholate, the SEC profiles of TLP were practically indistinguishable. At these low cholate concentrations there were modest changes in the size of LDL, whose elution volume decreased as a function of increasing initial cholate concentrations. Higher cholate concentrations produced larger shifts in the LDL peak so that at 360 mM, the LDL peak was barely discernible. Detergent perturbation also produced profound changes in the elution profile of HDL. As the initial cholate concentration increased, a new peak appeared in the chromatogram at fraction 22 and the major peak, corresponding to HDL, shifted to an earlier elution volumes that correspond to a higher particle weight (FIG. 5). Gradient gel electrophoresis (4-20% acrylamide) under non denaturing conditions corroborated the SEC data and showed that detergent perturbation increased the size of the HDL. The shift in the HDL peak and the magnitude of the peak with an elution volume of 34 mL was greatest at 90 mM cholate; at 180 mM cholate, the two well resolved peaks were replaced by a broad band of at least three components, which declined in magnitude when the initial cholate concentration was increased to 360 mM. Comparison of the magnitude of the peak with an elution volume of 34 mL with the initial cholate concentration reveals a small but distinct change just above the CMC (FIG. 6), suggesting that micelle formation plays a role, in the remodeling of HDL and release of apo A-I.

### EXAMPLE 12

### TLP Dose Dependence of Detergent Perturbation

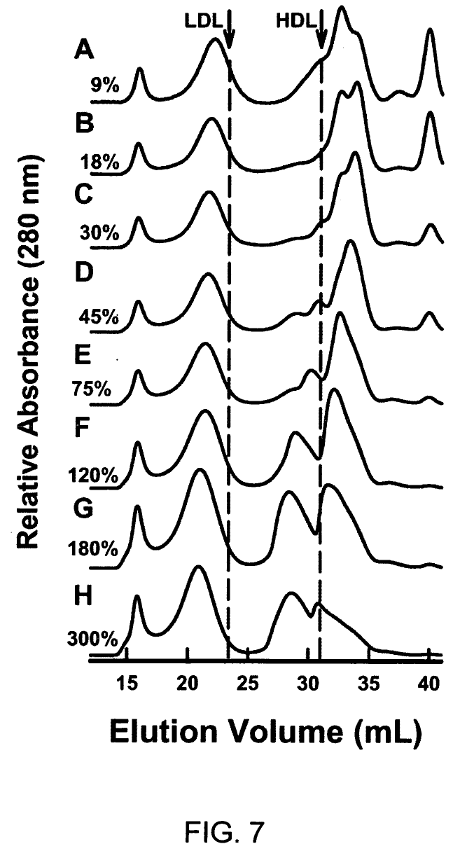

The solubilization power of a given cholate concentration on the distribution of lipoproteins was tested by using a constant dose of cholate while varying the TLP concentration from 9 to 300% of the plasma concentration with 100% being equal to the TLP protein concentration of the starting plasma (1.95 mg/mL TLP-protein). As shown in FIG. 7, the distribution of lipoprotein absorbance showed a slight shift in the peak for LDL at all TLP to cholate ratios. In contrast, the distribution of absorbance in the vicinity of the elution volume of HDL was different at all ratios tested with the lowest dose (9%) being associated with a shift in the absorbance toward a larger elution volume, which corresponds to a lower particle weight. As the TLP concentration was increased to 300%, the peak absorbance split into two peaks that were shifted toward a higher molecular weight. SEC after detergent perturbation of TLP at very high concentrations, ˜40 mg/mL protein, revealed a profile similar to that in FIG. 2E, except that the late eluting peak containing apo A-I is absent. Centrifugation of the detergent treated, dialyzed sample prior to SEC sedimented material that was not readily soluble in TBS but dissolved in 3 M guanidine hydrochloride. SDS PAGE analysis showed that this precipitate contained apo A-I as the sole protein. Thus, detergent perturbation at high TLP concentrations releases apo A-I that precipitates from solution upon detergent removal.

### EXAMPLE 13

### Effect of Detergent Perturbation at Various Dilutions at a Constant TLP/Cholate Ratio

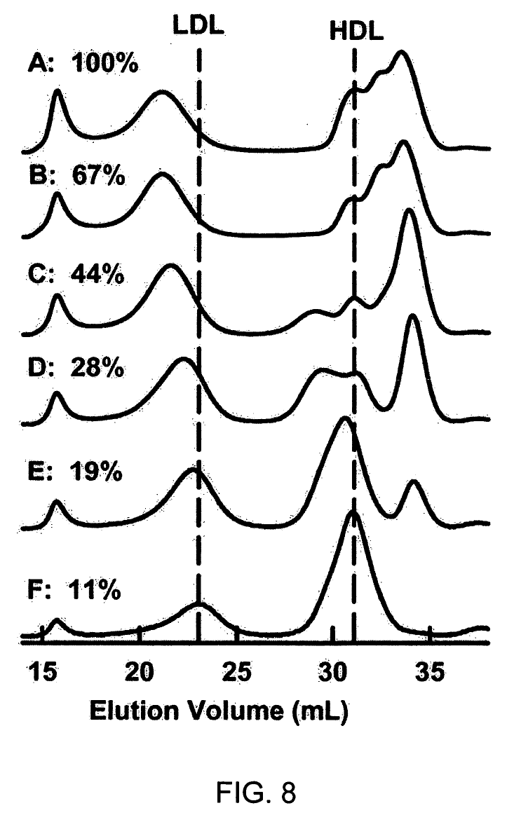

Various concentrations of TLP and cholate were combined while maintaining the same TLP/cholate ratio. The TLP was first diluted with that amount of TBS required to give each sample the same final concentration after which cholate was added. The samples were then dialyzed and analyzed by SEC on Superose HR6 as described above. With a decrease in the concentration of the sample from 100% to 11% of plasma TLP-protein concentration (1.95 mg/ml), the peak corresponding to LDL shifted to larger elution volumes, i.e. lower particle weights (FIG. 8). At 100% plasma TLP, the late eluting peak was composed of three poorly resolved peaks that collapsed to a single peak at 11% plasma TLP. At 11% TLP (0.2 mg/mL protein; 20 mM cholate), the Superose HR6 profile was indistinguishable from that of TLP without detergent perturbation (FIG. 1A).

### EXAMPLE 15

### Effect of HDL/LDL Ratio on Detergent Perturbation

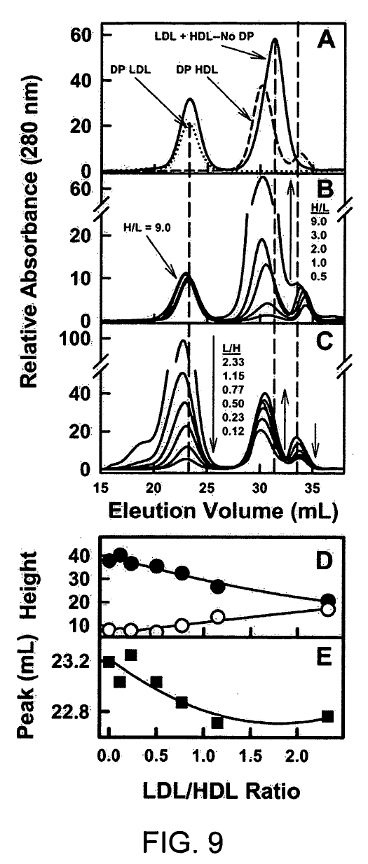

The changes in the SEC profile of LDL and HDL after detergent perturbation suggested that there may be a transfer of mass between these two particles. FIG. 9A compares the elution profiles of HDL and LDL after detergent perturbation with that of LDL+HDL without detergent perturbation. As with TLP, detergent perturbation of isolated HDL splits the HDL peak into two species, one eluting earlier and the other later than HDL. In contrast, detergent perturbation of isolated LDL had no effect on its elution profile. In both instances, detergent perturbation was conducted at approximately the plasma concentrations of LDL and HDL (0.3 and 1.3 mg/mL protein, respectively). The effects of HDL to LDL ratio on detergent perturbation were tested by varying HDL concentration at constant LDL concentration (FIG. 9B) and vice versa (FIG. 9C). Qualitatively the effects were similar. In both instances, the HDL peak split into two components and the LDL peak shifted to an earlier elution volume. However, according to FIG. 9B the largest fraction of protein appearing in the late HDL peak (lipid-free apo A-I) was at the lowest HDL/LDL ratio (0.5) with the fraction of total absorbance in the late peak decreasing as the HDL/LDL ratio was increased. The LDL peak was shifted only slightly even at the highest HDL/LDL ratio (9.0). At constant HDL concentration, the effects of detergent perturbation in the presence of increasing concentrations of LDL revealed systematic changes in the elution profiles. As the LDL/HDL ratio increased, the magnitude of the peak for HDL, which was shifted to earlier elution volumes, decreased while the peak for lipid-free apo A-I increased (FIG. 9 C, D). Concurrently, LDL eluted earlier, an effect that was dose dependent (FIG. 9 C, E). Thus, the increased lipid-free apo A-I produced with increasing LDL/HDL ratios correlated with an increase in the size of both LDL and the amount of large HDL.

### EXAMPLE 16

### Generalizing Detergent Perturbation

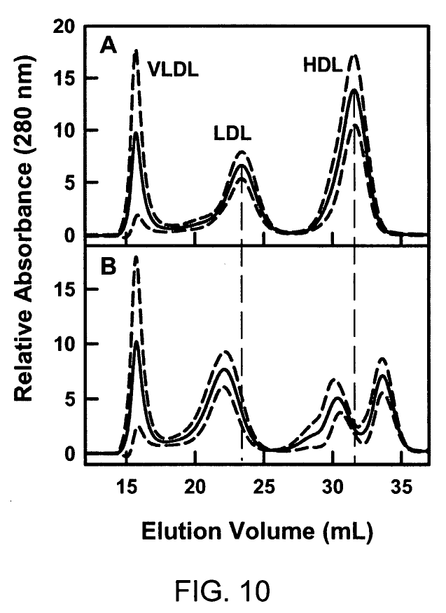

The effects of detergent perturbation on the plasma TLP from of eleven volunteers was tested to determine whether the observed effects of this process could be generalized. Based on the lipid data in Table 2, the mean SD plasma lipid levels for the donors were total cholesterol=147.1±26.0 mg/dL, triglyceride=92.3±52.7 mg/dL, HDL-C=35.8±9.0 mg/dL, and LDL-C=92.9±19.5 mg/dL. The TLP concentration used for detergent perturbation was adjusted to the original plasma lipoprotein level of each donor. The SEC of all eleven TLP before detergent perturbation revealed three peaks corresponding to VLDL, LDL, and HDL with elution volumes of ˜16, ˜23, and ˜32 mL, respectively; detergent perturbation shifted both LDL and HDL peaks to earlier elution volumes in all ten TLP samples with concurrent appearance of a peak corresponding to apo A-I (FIG. 10).

TABLE 2
Lipid Values for TLP Donors

| | | Plasma Analyte Concentration (mg/dL) | | | |
|---|---|---|---|---|---|
| Volunteer | | Total | | | |
| # | (Gender) | Cholesterol | TG | HDL-C | LDL-C |
| 1 | (M) | 165 | 52 | 49 | 106 |
| 2 | (M) | 163 | 167 | 36 | 94 |
| 3 | (M) | 127 | 44 | 39 | 79 |
| 4 | (M) | 185 | 61 | 46 | 127 |
| 5 | (M) | 127 | 185 | 26 | 64 |
| 6 | (M) | 151 | 52 | 34 | 107 |
| 7 | (M) | 116 | 101 | 23 | 73 |
| 8 | (M) | 192 | 151 | 45 | 117 |
| 9 | (F) | 137 | 58 | 38 | 87 |
| 10 | (M) | 133 | 100 | 23 | 90 |
| 11 | (F) | 122 | 44 | 35 | 78 |
| Mean ± | | 147.1 ± | 92.3 ± | 35.8 ± | 92.9 ± |
| SD | | 26.0 | 52.7 | 9.0 | 19.5 |

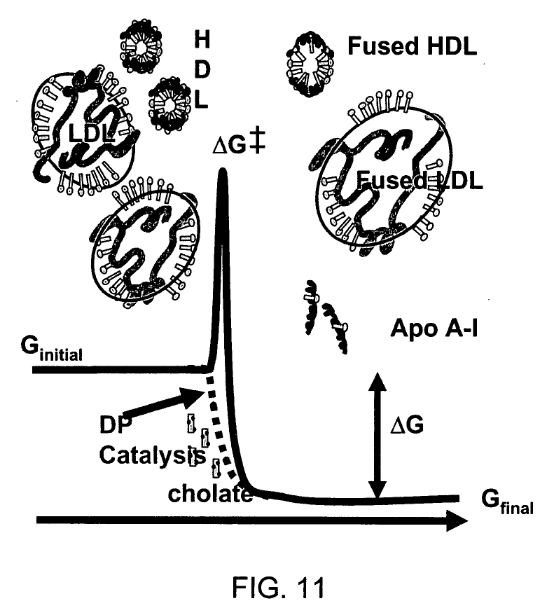

As summarized in the model in FIG. 11, detergent perturbation catalyzes the fusion of both LDL and HDL with the concomitant release of lipid-poor apo A-I, an effect that is dose dependent with respect to LDL concentration. Still further, apo B-100 and only apo B-100 is associated with LDL and its fusion product; similarly, apo A-I is only associated with lipid-poor apo A-I, HDL and its fusion product. Thus, the fusion appears to occur only between like species.

It is evisioned that these new species derived from lipoprotein fusion are cardioprotective and that their interactions with cholesterol transporters and hepatic lipoprotein receptors enhance reverse cholesterol transport by known or alternative pathways that would be uniquely determined by the structures of the fused LDL and HDL. Fusion, is an important step in the normal processing of human plasma HDL. In vitro HDL fusion can be induced by simple chemicals and by physiologically relevant reagents and comparison of products formed by these disparate reagents reveals clues to the fusion mechanism.

It is also envisioned that the compositions and sizes of the detergent perturbation-derived lipoproteins are controlled by two opposing forces that are determined by the hydrocarbon-like core lipids, CE and TG, and the surface components, mostly phospholipid and apolipoproteins. First there is the drive to minimize the neutral lipid-water interface. This may occur by forming single phase. On the other hand, there is an opposing force, which is to satisfy the amphiphilic properties of phospholipids and apoproteins. This can be achieved either as a mixed monolayer on the surface of a neutral lipid particle or as a distinct particle composed of an apo and phospholipids. Reduced availability of the phospholipids and apos that form the mixed monolayer barrier between the neutral lipid core and the surrounding aqueous phase would require the formation of larger particles. Thus, detergent perturbation first catalyzes the transfer of LDL- and HDL-phospholipids to cholate micelles resulting in the depletion of the lipoprotein surface of phospholipids the least lipophilic apo, apo A-I desorbs into the surrounding aqueous phase. As the cholate is removed, the phospholipids associate with apo A-I to give lipid-poor apo A-I. With a major fraction of the surface components of LDL and HDL transferred to lipid-poor apo A-I, the HDL and LDL fuse until they reach a size that permits the remaining apos and phospholipids to cover the neutral lipid-water barrier.

As a probe of macromolecular structure and stability, detergent perturbation of TLP offers several advantages over other methods. It is a simple method of studying HDL fusion and the concomitant formation of lipid-poor apo A-I in a way that does not require other plasma mediators, LCAT, or PLTP, which can be isolated only in small quantities by laborious multi-step procedures. Detergent perturbation does not break covalent bonds or use harsh conditions such as chemical denaturation or heating to high temperatures. Detergent perturbation by dilution is rapid (FIG. 1D) and provides a means of studying HDL fusion and the release of lipid-poor apo A-I in real time. Finally, detergent perturbation can be used on a large scale to form fused HDL and lipid-poor apo A-I for more detailed study of their structure and properties and their interactions with cellular lipid transporters and lipoprotein receptors.

### EXAMPLE 17

### SR-BI-Mediated Cholesterol Transport

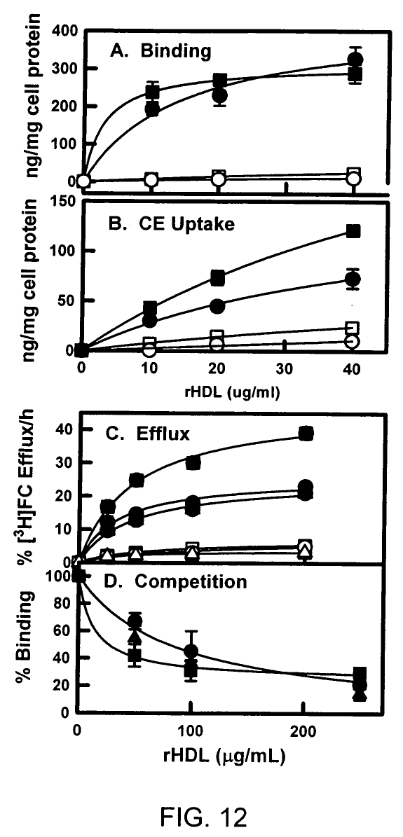

SR-B1, is a cell surface receptor that has a major role in both cholesterol efflux and the selective uptake of HDL-lipids (vide supra). Various cells have been used as models of these two components of RCT, including adrenal cells, We have used SR-BI-expressing (SR-BI+/+) CHO cells to test the effects of various rHDL on cholesterol efflux, selective uptake, and binding; LDL-receptor negative CHO cells (ldlA) expressing SR-BI and control ldlA CHO cells, which were kindly provided by Monty Krieger (Whitehead Institute), have been described (Acton et al., 1996). The inventors have used SR-BI+/+ cells to measure selective CE uptake, cholesterol efflux, and cell binding, both directly and by competition with rHDL (apo A-I+POPC, 1/100). rHDL composed of POPC and apo A-I, apo A-II, and reduced apo A-II were compared. In each case, the measurements were collected on control ldlA which do not expresss SR-BI+/+ (FIG. 12). These data show that relative to control cells, a] rHDL binding, b] selective CE uptake, and c] cholesterol efflux to rHDL are enhanced in SR-BI+/+cells (FIGS. 12 A-C, respectively), binding, selective uptake, and cholesterol efflux are greater for apo A-I-containing rHDL than for apo A-II-containing rHDL (FIGS. 12 A-C, respectively), and reduction of apo A-II in rHDL has no effect on cholesterol efflux (FIG. 12 C), and apo A-I rHDL completes for binding to SR-BI with rHDL containing apo A-I, A-I and reduced A-II (FIG. 12 D).

### EXAMPLE 18

### Efflux Via SR-BI

Measurements of cholesterol efflux are essentially according to standard methods (Yancey et al., 2000, Williams et al., 2000, Rothblat et al., 1986, Thuahnai et al., 2001, Rodrigueza et al., 1999, de la Lera-Moya et al., 1999). Briefly, after purifying [1,2-³H]cholesterol by silica gel chromatography to remove polar impurities, the pooled fractions of pure cholesterol are reduced to dryness under a stream of nitrogen and resolubilized in DMSO. The DMSO solution of labeled cholesterol is rapidly mixed with heat-inactivated (1 hour @ 55° C.) serum or serum containing medium (F12) at the rate of 10 μCi/mL, filtered (0.45 μm) for 48 hours at 37° C. SR-BI+/+ and ldlA cells are grown to ˜80% confluence in 35 mm dishes in medium containing 5% heat inactivated calf serum. Cells are then incubated with the labeled serum-containing medium for 48 hours and then for an additional 24 h in medium containing fatty acid-free bovine serum albumin to ensure equilibration of labeled cholesterol into all intracellular pools. Labeling is validated by thin layer chromatography separation of free and esterified cholesterol, which are quantified by transferring the spots on the plate to scintillation vials and counting. Cell monolayers are washed twice with medium containing 1% albuminin after which medium (1-mL) containing FR186054, an inhibitor of acyl-CoA:cholesterol acyltransferase,84 and acceptors (rHDL, HDL, LDL, and DTR-LDL and -HDL fractions isolated from TLP by Superose 6 chromatography) prewarmed to 37° C. are added to triplicate plates and control plates without cells. After incubation for 2 hours, the medium is collected and the cells are extracted into 2-propanol:hexane (3/2); two aliquots of both extract (40%) and medium (0.4-mL) are collected. One of each is analyzed by liquid scintillation counting. Efflux is expressed as the percent of total counts in free cholesterol (cell-associated counts+medium) found in the medium. The other two aliquots are analyzed by GC-MS to determine net transfer, i.e. loss by cells and gain by acceptors.

### EXAMPLE 19

### Efflux Via ABCA1

J774 mouse macrophages are grown at 37° C., 5% CO₂ in RPMI 1640 with 10% FBS supplemented with 50 μg/ml gentamicin. Cells are seeded into 12- or 24-well plates or 35-mm dishes and grown to 80-90% confluence. Monolayers are washed with MEM-Hepes and incubated for 24 h in RPMI 1640 containing [1,2-³H]cholesterol (2 μCi/well), 1% FBS and FR186054 to inhibit ACAT. Labeled cells are washed and, unless otherwise indicated, incubated with 0.2% BSA in RPMI 1640 with or without 0.3 mM cpt-cAMP for 12 hours, after which some wells are washed with PBS, dried, and extracted with 2-propanol to give the baseline (t0) values for total cellular [1,2-³H]cholesterol content. Cpt-cAMP-treated and control monolayers are washed with MEM-Hepes and incubated for 4 h in the presence and absence of cholesterol acceptors, after which the media is collected and filtered (0.45 μm) to remove cells and cell debris. Efflux and net transfer are determined as described above.

### EXAMPLE 20

### ABCA1 Expression

Parallel cultures, which are incubated with the same media without labeling, are used to determine ABCA1 protein and mRNA. Unlabeled monolayers grown in 100-mm dishes are harvested for total RNA isolation with TriZol™ reagent or for protein analysis with lysis buffer (50 mM Tris-HCl, 150 mM NaCl, 0.02% sodium azide, 1% Nonidet P-40, 2 mM Pefabloc, 2 μg/ml aprotinin, pH 8.0). Cells are scraped from the monolayers, repeatedly pipetted, and then centrifuged at 12,000×g for 4 min to pellet nuclei and debris. Lysates for mRNA and protein supernatants are stored at 70° C. until analyzed.

### EXAMPLE 21

### Efflux by Via ABCG1 Expression

Parallel HEK293 cells are grown in DMEM+10% fetal bovine serum and plated in 24-well plates (0.3×10⁶ cells/ml) and allowed to adhere overnight. LipofectAMINE 2000 (Invitrogen) is used to transiently transfect triplicate wells of Hek293 cells with the empty pCMX vector (1 μg/well) or the pCMX vector containing a cDNA encoding a specific ABCG1 isoform. Cells transfected with plasmids encoding green fluorescent protein and pCMX are used to determine transfection efficiency.

### EXAMPLE 22

### Preparation of PC-Rich Lipoproteins

For PC enrichment, POPC, which is cholesterophilic (Niu et al., 2002) an LCAT substrate (Pownall et al., 1982) and a natural phospholipid species of plasma lipoproteins, was used (McKeone et al., 1997). TLP and 465 mM sodium cholate were mixed on ice with various amounts of POPC to achieve final concentrations between 65 and 90 mM cholate and the original plasma TLP concentration, ˜2 mg/mL protein. The samples were dialyzed (Spectra/Por, r=7.3 mm, molecular weight cutoff for retention ˜6,000-8,000) for 48 hours against a 1000-fold excess of TBS at 4° C. with a change of buffer at 24 hours. Lipoproteins were analyzed by size exclusion chromatography (SEC) (Pownall 2005).

### EXAMPLE 23

### Lipoprotein Cholesterophilicity

In triplicate, TLP (1 mL, 7.8 mg/mL TLP-protein) was mixed with 0, 0.165, 0.33, and 0.5 mL POPC (20 mg/mL), 0.775 mL sodium cholate (465 mM), and sufficient TBS to give a final concentration of 1.95 mg/mL TLP-protein. Control samples were prepared without detergent or POPC. The samples were dialyzed as described above. Cholesterophilicity was determined by measuring cholesterol partitioning between lipoproteins and 2-hydroxypropyl-cyclodextrin (CDX).21 At room temperature, 400 μL of each sample were mixed with 200 mM CDX (75 μL) and 25 μL TBS. The samples were incubated for three hours and transferred to Microcon ultracentrifugal filters with an exclusion limit of 30 kDa and centrifuged for 10 min at 12,000 rpm in an Eppendorf microfuge. Aliquots of the retentate and filtrate were analyzed for cholesterol, protein, and phospholipid. The partition coefficient Kp for the distribution of cholesterol between TLP and CDX was calculated from
Kp={FC_{Bound}×[CDX]}.{FC_{CDX}×TLP_{Pro}} Equation 1

where TLP_{Pro} is the initial TLP-protein concentration (mg/mL), FC_{TLP}, FC_{CDX}, and FC_{Bound}, respectively are the free cholesterol concentrations in the retentate, filtrate, and bound to TLP with FCBound calculated as FC_{TLP.}−FC_{CDX}.

### EXAMPLE 24

Association of POPC with Lipoproteins

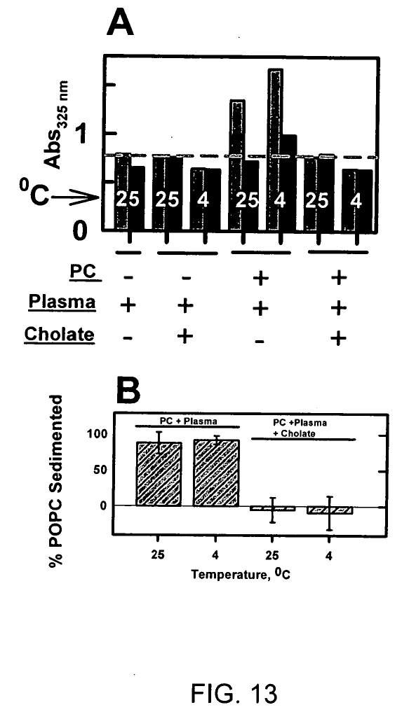

Association of POPC with lipoproteins in whole plasma was assessed with a radioactive tracer. [³H]POPC (10 mg/mL; 0.4 μCi/mg) was suspended in TBS (2.5 mL) at room temperature and vortexed. Human plasma, [³H]POPC liposomes and sodium cholate were mixed and dialyzed at room temperature or 4° C., 17 after which the turbidity of each sample was measured by absorbance at 325 nm. The samples were assayed for radioactivity before and after ultracentrifugation at 10,000×g for 10 min, which sediments the liposomes (>90%). Plasma alone had an absorbance of ˜0.7 (FIG. 13), which represents the sum of the absorbance and light scattering by macromolecules. Neither the addition of cholate nor sedimentation greatly affected the turbidity at 4 or 25° C. However, at both 4 and 25° C., addition of POPC liposomes to plasma increased the turbidity. Centrifugation, which sediments the liposomes, reduced the absorbance to a value similar to that of plasma alone. Those samples that were incubated with sodium cholate and then dialyzed showed no increase in turbidity when compared to plasma alone and centrifugation had no additional clearing effect, suggesting that cholate “catalyzes” POPC incorporation into macromolecules that were too small for low speed sedimentation. Analysis of the radiolabeled POPC confirmed this (FIG. 13B); centrifugation sedimented ˜90% of the POPC that was incubated with plasma only. In contrast, little or no [³H]POPC sedimented from samples that were incubated with cholate before dialysis. Thus, cholate treatment (detergent perturbation) effects liposomal POPC incorporation into lipoprotein particles in whole plasma.

Other tests were conducted with the TLP fraction of whole plasma, which contains VLDL, LDL, and HDL. TLP was adjusted to its original plasma concentration (TLP-protein=2.0 mg/mL) and [3H]cholate and graded amounts of POPC liposomes were added and dialyzed. [³H]Cholate disappeared exponentially during dialysis. The respective half times for this process were 2.7, 3.0, and 3.5 h, for cholate, TLP, and TLP+1.6 mg/mL POPC. A similar experiment conducted with [³H]POPC, cholate, and TLP showed quantitative recovery (˜99%) of POPC in the TLP after DP and dialysis.

### EXAMPLE 25

### Incorporation of POPC in Lipoprotein Fractions

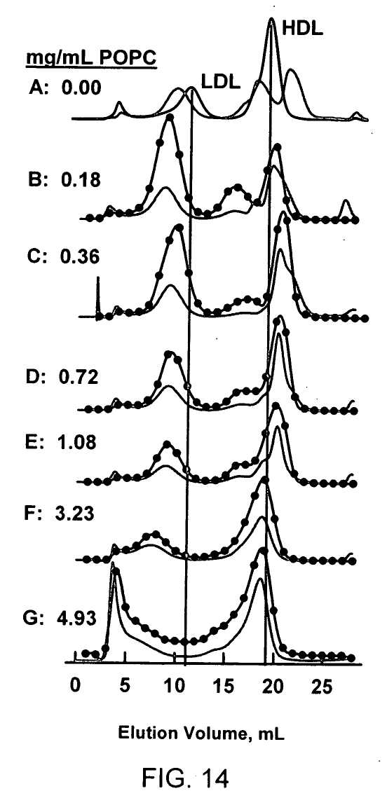

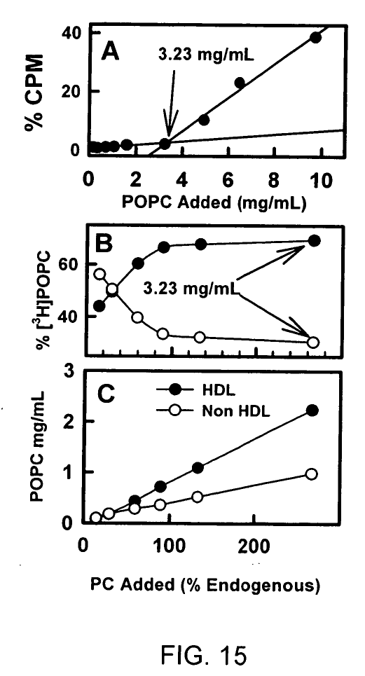

Next, the distribution of incorporated POPC among the lipoprotein fractions was determined. TLP was mixed with sodium cholate and [³H]POPC, dialyzed, and analyzed by SEC, which separates control TLP into VLDL, LDL, and HDL (FIG. 14A, dashed gray curve). DP of TLP alone shifted the LDL peak to an earlier elution time and split HDL into two peaks, an early one corresponding to fused HDL and a late one, which is lipid-free apo A-I17 (FIG. 14A, solid gray curve). With POPC addition, the LDL particle size increased while the two peaks for HDL (FIG. 14A) collapsed into a single peak that was relatively symmetrical at higher concentrations of POPC (FIG. 14B-14F); addition of >4.93 mg/mL POPC ablated the LDL peak and most of the additional POPC appeared in the void volume (FIGS. 14 and 15A). The amounts of [³H]POPC associated with LDL and HDL were dose-dependent. At low concentrations, most of the [³H]POPC associated with LDL (FIGS. 14B and 15B), but as the [³H]POPC concentration increased, the percent POPC associated with LDL and HDL decreased and increased, respectively (FIGS. 14C-F and 15B, C). Thus, LDL has a higher affinity for POPC than HDL but HDL has a greater capacity.

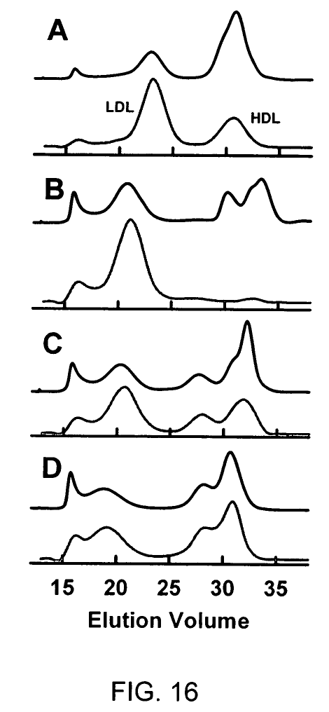

According to SEC of control TLP, at equilibrium ˜70% and ˜30% of the radiolabeled cholesterol was associated with LDL and HDL, respectively (FIG. 16A). Following DP, [³H]cholesterol was almost exclusively (˜95%) associated with LDL (FIG. 16B); the HDL peak was bimodal, appearing as early and late eluting fractions. However, as the amount of added PC was increased, the fraction of [³H]cholesterol associated with HDL increased (FIGS. 16C and D, respectively).

### EXAMPLE 26

### Effects of Enriching LDL and HDL with POPC

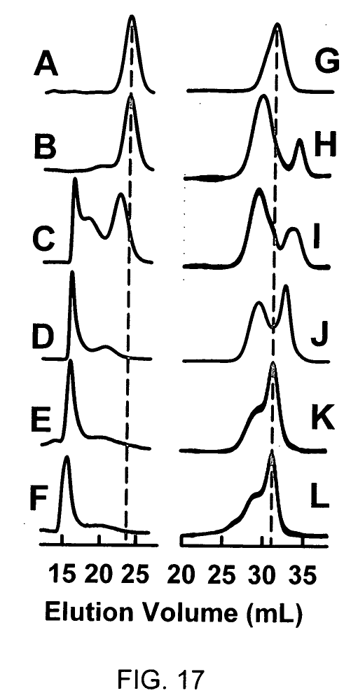

The effects of enriching isolated LDL and HDL with POPC were also studied. Detergent treatment alone had no effect on isolated LDL but HDL split into two peaks. Whereas all the samples prepared from HDL were optically clear, at higher POPC concentrations, the LDL samples were cloudy. After filtration and centrifugation (15 minutes at 10,000×g), samples corresponding to FIGS. 17A-D lost little or no phospholipid whereas those corresponding to FIGS. 17E and 17F, respectively, lost 25 and 70 percent of the phospholipid. Addition of small amounts of phospholipid to LDL formed products that eluted in the void volume and the LDL peak disappeared as a function of increasing phospholipid. This is consistent with a low capacity of LDL for exogenous POPC. In contrast, following addition of POPC to HDL, the two peaks observed with detergent alone coalesced into one peak that was similar to that of HDL; HDL had a higher capacity for incorporation of exogenous POPC, up to ˜2.5 times the initial PL concentration.

### EXAMPLE 27

### Cholesterophilicity of TLP

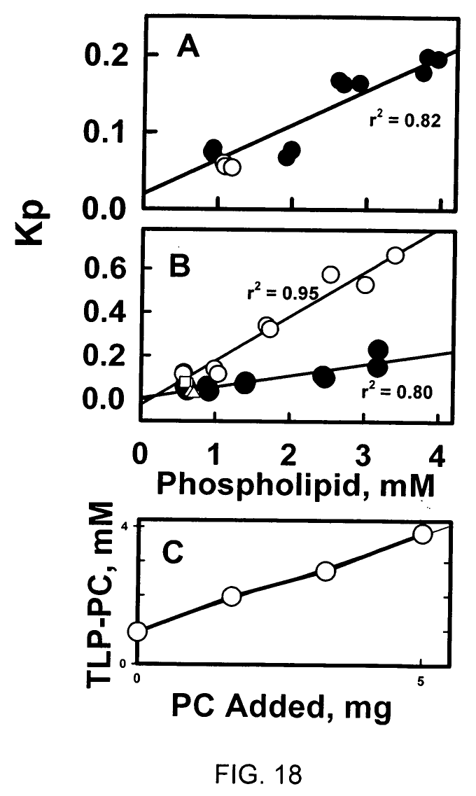

The cholesterophilicity of TLP was a linear function of the amount of added POPC (FIG. 18A, insert). Moreover, the amount of cholesterol associated with TLP increased as a positive function of the amount of POPC in the TLP (FIG. 6A). The linearity of the curve is consistent with each increment of POPC added contributing equally to its cholesterophilicity. The Y-intercept for the plot is nearly zero suggesting similar contributions of endogenous and exogenous PC to cholesterophilicity. Isolated LDL and HDL were enriched with POPC by DP and the cholesterophilicities measured by CDX partitioning (This was done on the same samples shown in FIG. 17 but was performed before filtration and centrifugation). These data (FIG. 18B) show a POPC dose-dependent increase in the cholesterophilicities of LDL and HDL with the effects of added POPC on KP for LDL being greater than that of HDL. The cholesterophilicity of LDL was higher than that of HDL at all phospholipid concentrations, including those for native LDL (0.075±0.003) and HDL (0.027±0.003). However, according to SEC FIG. 17 (A-F), much of the increase in KP for LDL with added POPC is due to non-LDL associated POPC and to material eluting in the void volume. The slopes for cholesterophilicity vs. TLP- and HDL-phospholipid were similar (0.042 and 0.052, respectively) suggesting that most of the POPC-dependent differences in TLP cholesterophilicity is due to HDL.

### REFERENCES CITED

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
Acton et al., Science. 1996 271:518-20.
Albers et al., Methods Enzymol. 1986; 129:763-83.
Arai et al., J Biol. Chem. 1999 274:2366-2271.
Aron et al., J Biol. Chem. 1978 253:7220-6.
Barter P J, et al., Arterioscler Thromb Vasc Biol. 2003 Feb. 1; 23(2):160-7.
Bielicki et al., J Lipid Res. 1992 33:1699-709.
Blanche et al., Biochim Biophys Acta. 1981 665:408-19.
Bortnick et al., J Biol. Chem. 2000 275:28634-40.
Braun et al., Proc Natl Acad Sci USA. 2003 100:7283-7288.
Brewer et al., Arterioscler Thromb Vasc Biol. 2004 24:1755-60.
Covey et al., Arterioscler Thromb Vasc Biol. 2003 23:1589-1594.
Davidson et al., J Biol. Chem. 1995 270:5882-90.
Day, 1975 Artery 1:1-2.
de Beer et al., J Biol. Chem. 2001 276:15832-9.
de Beer et al., J Lipid Res. 2001 42:309-313.
de Beer et al., J Lipid Res. 2004 45:706-15.
de la Llera-Moya et al., J Lipid Res. 1999 40:575-80.
Fielding and Fielding, Biochim Biophys Acta. 2001 1533:175-89.
Fournier et al., J Lipid Res. 1996 37:1704-11.
Francis et al., J Clin Invest. 1995 96:78-87.
Carey et al., Arch. Inter Med. 130:506-527 (1972).
Hjelm, Jr. et al., J. Phys. Chem. 96 (21):8653-8661 (1992)
Frick et al., N Engl J. Med. 1987 Nov. 12; 317(20):1237-45.
Glomset, J Lipid Res. 1968 9:155-67.
Grundy S M, et al.,; Coordinating Committee of the National Cholesterol Education Program. Implications of recent clinical trials for the National Cholesterol Education Program Adult Treatment Panel III Guidelines. J Am Coll Cardiol. 2004 Aug. 4; 44(3):720-32.
Hara et al., J Biol. Chem. 1991 266:3080-6.
Havel R J et al., Lipoproteins and Lipid Transport. In The Metabolic Control of Disease (P. K. Bondy and L. E. Rosenberg, Eds.) 393-494. Saunders Publishing, Philadelphia Pa.
Huszar et al., Arterioscler Thromb Vasc Biol. 2000 20:1068-1073.
Huttunen et al., Helsinki Heart Study. New perspectives in the prevention of coronary heart disease. Drugs. 1988; 36 Suppl 3:32-6.
Ji et al., J Biol. Chem. 1999 274:33398-33402.
Jian B, et al., J Lipid Res. 1997 38:734-44.
Jian B, et al., J Lipid Res. 1997 38:734-44.
Johnson et al., J Biol. Chem. 1986 261:5766-76.
Kozarsky et al., Arterioscler Thromb Vasc Biol. 2000 20:721-727.
Kozarsky et al., Nature. 1997; 387:414-417.
Liadaki et al., J Biol. Chem. 2000 275:21262-21271.
Linsel-Nitschke P, Tall A R. Nat Rev Drug Discov. 2005 March; 4(3):193-205.
Liu et al., J Biol. Chem. 2002 277:21576-21584.
Lund-Katz et al., Biochemistry. 1988 27:3416-23.
Lund-Katz et al., Lipids. 1996 31:1107-13.
Lusa et al., Biochem J. 1996 313:275-82.
Manninen et al., JAMA. 1988 260:641-51.
Markwell et al., 1978 Anal. Biochem. 87, 206-210.
Mason et al., Anal Biochem. 1981 113:96-101.
Almgren et al., Colloid Polym. Sci. 273:2-15 (1995).
Nivaggioli et al., Langmuir. 11 (3):730-737 (1995)
Alexandridis et al., Langmuir. 11:1468-1476 (1995)
Matz and Jonas, J Biol. Chem. 1982 257:4535-40.
Matz et al., J Biol. Chem. 1982 257:4541-6.
Methods in Enzymology Volumes 128 and 129, published in 1986.
Nakamura et al., J Biol. Chem. 2004 279:45980-9.
Nanjee et al., Arterioscler Thromb Vasc Biol. 1999 19:979-89.
Nanjee et al., J Lipid Res. 2001 42:1586-93.
Nissen et al., JAMA. 2003 290:2292-300.
Niu et al., Biophys J. 2002 83:3408-15.
Okuhira et al., J Lipid Res. 2004 45:645-52.
Patel et al., Lipids 1979 14:816-818.
Phillips et al., Atherosclerosis. 1998 April; 137 Suppl:S13-7.
Phillips et al., Biochim Biophys Acta. 1987 906:223-76.
Picardo et al., Arteriosclerosis. 1986 6:434-41.
Pilon et al., Arterioscler Thromb Vasc Biol. 2000 20:1074-1081.
Pownall et al., Atherosclerosis. 1999 143:285-97.
Pownall et al., Biochim Biophys Acta. 1982 713:494-503.
Pownall et al., J Biol. Chem. 1985 260:2146-52.
Qu et al., J Lipid Res. 1995 36:967-74.
Rao et al., Biochemistry. 1997 36:3645-53.
Rigotti et al., Endocr Rev. 2003 24:357-387.
Rigotti et al., Endocr Rev. 2003 24:357-387.
Rodrigueza et al., J Biol. Chem. 1999 274:20344-50.
Rothblat et al., Methods Enzymol. 1986; 129:628-44.
Sehayek et al., Proc Natl Acad Sci USA. 1998 95:10194-10199.
Shumaker and Puppione, 1986 Methods Enzymol. 128:155-169.
Tanaka et al., Bioorg Med. Chem. 1998 6:15-30.
Temel et al., J Biol. Chem. 2002 277:26565-26572.
Temel et al., J Biol. Chem. 2003 278:4792-4799.
Thuahnai et al., J Biol. Chem. 2001 276:43801-8.
Thuahnai et al., J Lipid Res. 2003 44:1132-1142.
Tollefson and Albers, Methods Enzymol. 1986; 129:797-816.
Ueda Y et al., J Biol. Chem. 2000 275:20368-20373.
Ueda Y Royer et al., J Biol. Chem. 1999 274:7165-7171.
Van Eck et al., J Biol. Chem. 2003 278:23699-23705.
Varban et al., Proc Natl Acad Sci USA. 1998 95: 4619-4624.
Wang et al., J Biol. Chem. 1998 273:32920-32926.
Wang et al., Proc Natl Acad Sci USA. 2004 101:9774-9.
Williams et al., J Biol. Chem. 2000 275:18897-18904.
Yancey et al., Arterioscler Thromb Vasc Biol. 2003 23:712-9.
Yancey et al., Biochemistry. 1995 34:7955-65.
Yancey et al., J Biol. Chem. 2000 275:36596-604.
Yang et al., J Biol. Chem. 1987 262:3086-91.
Zhong et al., J Clin Invest. 1996 97:2917-23.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of increasing the activity associated with a lipoprotein comprising the step of administering to a sample containing lipoprotein a composition comprising a detergent that increases the activity associated with the lipoprotein.

2. The method of claim 1 further comprising administering a phospholipid.

3. The method of claim 1, wherein the activity is cholesterol binding affinity, lecithin:cholesterol acyltransferase (LCAT) activity or a combination thereof.

4. The method of claim 1, wherein the activity is an increase in cholesterol esterification.

5. The method of claim 1, wherein the detergent is an anionic detergent, non-ionic detergent, a bile acid or a combination thereof.

6. The method of claim 1, wherein the detergent is sodium cholate.

7. The method of claim 2, wherein the phospholipid is phosphodidylcholine.

8. The method of claim 2, wherein the phospholipid is lecithin.

9. The method of claim 1, wherein the lipoprotein is total plasma lipoproteins.

10. The method of claim 1, wherein the lipoprotein is a high density lipoprotein (HDL) or a low density lipoprotein (LDL).

11. The method of claim 2, wherein the amount of phospholipid is in the range of about 10 mg/liter of sample to about 10 g/liter of sample.

12. The method of claim 14, wherein the ratio of detergent to phospholipid is about 1:10 to about 500:1.

13. The method of claim 2, wherein increased lipoprotein associated activity increases lipid metabolism, decreases hyperlipidemia or a combination thereof.

14. The method of claim 13, wherein increased lipoprotein associated activity decreases atherosclerosis in a human.

15. A method of increasing reverse cholesterol transport in a subject comprising the steps of
i) obtaining a sample from the subject;
ii) administering to the sample a composition comprising a detergent and a phospholipid; and
iii) administering the sample of step ii) to the subject, wherein the administered sample increases reverse cholesterol transport in a subject.

16. A method of increasing lipid metabolism in a subject suffering from hyperlipidemia comprising the steps of
obtaining a blood sample from the subject;
treating the blood sample with a detergent and a phospholipid to the blood sample;
administering the treated blood sample to the subject, wherein the treated blood sample increases lipid metabolism in the subject.

17. The method of claim 16, wherein prior to the administering step the treated sample is dialyzed.

18. A method of regulating the levels of cholesterol in a subject comprising the steps of:
i. measuring the levels of cholesterol in a subject, if the levels of cholesterol are above normal, obtaining a treatment sample from the subject;
ii. administering to the treatment sample a composition comprising a detergent and a phospholipid;
iii. administering the sample of step ii to the subject; and
v. repeating steps i-iiii until the cholesterol level of the subject is at a satisfactory level.

19. The method of claim 1, wherein the composition is administered to a subject in need thereof to treat a cardiovascular disease.

20. The method of claim 19, wherein the composition further comprises a phospholipid.

21. The method of claim 19, wherein administering the composition comprises treating a blood sample with the composition ex vivo prior to the administering the composition to the subject.

22. The method of claim 19, wherein the cardiovascular disease is atherosclerosis.

23. The method of claim 19, wherein the composition increases the process of reverse cholesterol transport (RCT).

24. The method of claim 23, wherein RCT is increased by increasing the cholesterolphilicty of a lipoprotein.

25. The method of claim 24, wherein the lipoprotein is high density lipoprotein (HDL).

26. The method of claim 24, wherein the lipoprotein is low density lipoprotein (LDL).

27. The method of claim 24, wherein RCT is increased by increasing the activity of lecithin:acyltransferase activity.
